SEQUENCE LISTING (1 sequence)

SEQ ID NO: 1 - protein, 30 aa, Artificial Sequence
  Heptad repeat sequence
MKQIEDKIEEILSKIYHIENEIARIKKLIK

(19) United States
(12) Patent Application Publication
(10) Pub. No.: US 2023/0243817 A1
(43) Pub. Date: Aug. 3, 2023

(54) USES, METHODS AND PRODUCTS RELATING TO OLIGOMERIC LIPOPOLYSACCHARIDE BINDING PROTEINS

(71) Applicant: UNIVERSITETET I OSLO, Oslo (NO)

(72) Inventors: Daniel HATLEM, Oslo (NO); Dirk LINKE, Oslo (NO); Stefanie Brigitte BARBIRZ, Berlin (DE)

(73) Assignee: UNIVERSITETET I OSLO, Oslo (NO)

(21) Appl. No.: 18/002,800

(22) PCT Filed: Jun. 24, 2021
(86) PCT No.: PCT/EP2021/067413

§ 371 Date: Dec. 21, 2022

(30) Foreign Application Priority Data
Jun. 25, 2020 (GB) 2009730.9

(51) Int. Cl. G01N 33/543; G01N 33/569

(52) U.S. Cl. CPC G01N 33/54353; G01N 33/569; G01N 2400/50

(57) ABSTRACT

Provided and described herein is the use of an oligomeric protein as a binding agent for binding to lipopolysaccharide (LPS), the oligomeric protein having a coiled coil structure comprising at least two monomer peptides, wherein each monomer peptide, which may be the same or different, is capable of forming an α-helix and comprises at least one core sequence having at least 60% sequence identity to the heptad repeat sequence of SEQ ID NO. 1. Also provide and described herein are methods of binding, detecting and removing LPS, and products comprising the oligomeric protein.

### TECHNICAL FIELD

Described herein is the use of an oligomeric protein having a coiled coil structure as a binding agent for binding to lipopolysaccharide (LPS). Also described herein are methods of binding, detecting and removing LPS, and products comprising the oligomeric protein.

### BACKGROUND

Lipopolysaccharides, also known as endotoxins (the terms “LPS” and “endotoxin” are used interchangeably herein), are an essential component of the outer membrane of Gram-negative bacteria. They are composed of three structural components: the lipid A moiety; the core oligosaccharide; and the O-antigen. The lipid A component comprises two phosphoglucosamine molecules with four O-linked and two N-linked acyl chains, which are embedded in the outer membrane of the bacteria and thus anchor the LPS into the bacterial membrane. The core oligosaccharide (COS) is a non-repeating structure made up of a variety of sugars, and is linked to the lipid A via a glycosidic bond. Finally, the O-antigen is a polymer made up of between 4 to 40 units of a four-sugar monomer repeat structure, with an average of 30 repeats present (Peterson and McGroarty, 1985). The O-antigen is linked to the penultimate sugar of the COS, at the opposite end to lipid A, though in some forms of LPS the O-antigen is absent. LPS lacking the O-antigen is traditionally termed ‘rough’ LPS, as opposed to the wild type ‘smooth’ LPS (Hitchcock et al, 1986).

The lipid A component of LPS, and the sugars most proximal thereto, are highly conserved across Gram-negative bacterial species, while the rest of the core oligosaccharide and the O-antigen are significantly less conserved, and can vary between bacterial species and even between serotypes (Bertani and Ruiz, 2018).

Endotoxins are extremely toxic to animals, particularly to humans, due to the propensity of lipid A to activate toll-like receptor 4, and thus induce an extreme immune reaction, which can cause sepsis and toxic shock in doses as low as 1 μg per kg body weight. Due to the omnipresence of Gram-negative bacteria in biological environments, endotoxins are a common impurity in the production of medicines, vaccines, and laboratory equipment and reagents. In view of the potential health impacts associated with endotoxin contamination, it is vital to remove, as far as is possible, any endotoxin that may be present in products intended for human consumption, prior to use.

Various methods and products for endotoxin removal are presently available. For laboratory use these include spin column filters or flow columns packed with a resin which is linked to one or more endotoxin-binding molecules. A sample can be applied to these filters or columns, and the endotoxin-binding molecules will bind to any endotoxins that are present, thus removing them from the sample. Known endotoxin-binding molecules that can be used in these products include the lipid A-binding antibiotic polymyxin B, and poly-lysine polymers, which bind endotoxins via electrostatic interactions. However, there are problems with these presently available endotoxin-binding molecules. Polymyxin B has a reported binding affinity only in the micromolar range, depending on the bacterial strain in question, which makes it unsuitable for binding to and removing low concentrations of endotoxin from a sample. Poly-lysine, meanwhile, is highly positively charged and thus interacts with the negatively charged phosphate groups of lipid A and the core oligosaccharide sugars in a non-specific manner. This mechanism of action is therefore not appropriate for use at all pH values or with all LPS types. Moreover, it may also interact with, and therefore remove, other negatively charged molecules which may be present in a sample.

Other methods include ion exchange chromatography. This is commonly used in the pharmaceutical industry for the purification of pharmaceutical products, and typically relies on electrostatic interactions between the negatively charged LPS and a positively charged immobilised ligand. However using ion exchange chromatography to remove endotoxins from highly charged samples can be problematic. For instance, if a sample contains highly positively charged particles, these particles will compete with the immobilised ligand to capture LPS. Conversely, if the sample contains highly negatively charged (non-LPS) particles, these particles will compete with LPS for binding to the immobilised ligand. In both cases, these unwanted reactions lower the efficiency of the LPS removal. The electrostatic interactions which underpin the ion exchange chromatography methods can also be disrupted in samples with high ionic strength, and thus these methods are not appropriate in all scenarios.

In addition to removing endotoxins from samples, it is also desirable to be able to detect endotoxins, so that therapeutic products, devices, reagents, etc. can be certified as “endotoxin-free”, and therefore safe to use in therapeutic applications. In this regard, the most common method of detecting endotoxins is currently the Limulus amebocyte lysate (LAL) assay. The LAL assay is approved by the FDA and EFSA for detection of endotoxins in medical and therapeutic products with a detection range down to 1 picomolar (0.1 EU/mL) concentration. This assay uses a lysate of amebocyte cells found in the blood of horseshoe crabs of the genus Limulus, which contains a complex mixture of proteins and enzymes. In particular, the LAL assay is based on the activity of the enzyme “(limulus clotting) factor C”, (commonly known simply as Factor C), which is triggered upon LPS binding. Factor C is a trypsin type serine protease that activates a complex cascade of downstream enzymatic reactions, which ultimately provide an indication of the presence of LPS. However, this reaction cascade can also be activated by beta-glucans, which are commonly found in a range of bacteria, fungi, and plants. Accordingly, beta-glucans can cause false positive results in LAL assays. Furthermore, the amebocyte lysate is very expensive to produce, and the current productions methods are not sustainable.

The overharvesting of horseshoe crabs has led to calls for the development of alternative methods for endotoxin detection. A similar assay has been developed which uses recombinantly expressed Factor C to cleave a chromogenic substrate, thus allowing LPS to be detected more directly. However, due to the complexity of the composition, the price is still high.

Moreover, the activity of Factor C can be easily disrupted, for example due to variations in temperature or pH, denaturing compounds such as organic solvents, urea, or strong detergents, and convention protease inhibitors. There can also be batch-to-batch variation between different Factor C preparations. This makes the enzyme difficult to work with, and means that the results that are obtained with LAL assays which rely on Factor C are often not particularly consistent or reproducible.

A further problem in the detection of LPS which affects all detection methods, is the tendency of LPS to aggregate. It is known in the art that endotoxin molecules tend to form aggregates in aqueous solutions. This aggregation is increased by the presence of cations (particularly divalent cations such as Ca²⁺ and Mg²⁺) in a solution, and also by the presence of detergents, which can form micelles around the LPS. This aggregation has the effect of reducing the amount of measurable LPS in solution, and therefore inhibits the detection of low concentrations of LPS. This effect is known as “LPS masking”, and can be caused by a wide range of different agents. For example, in blood samples, there are a multitude of compounds that can mask LPS, such as LPS binding proteins, anti-LPS antibodies, and divalent cations. In addition, endotoxin molecules from different bacterial sources can have different molecular weights, and can exhibit different aggregation behaviour, which results in variable results when measuring the same concentration of LPS from different sources. Accordingly, it may be useful to provide an improved LPS binding agent that can be used to remove or detect LPS.

### SUMMARY

The present inventors have developed a novel LPS binding agent, in the form of an oligomeric protein having an alpha-helical coiled-coil structure.

The new LPS-binding agent disclosed herein is based on the alpha-helical coiled-coil structure that can be found in the yeast transcription factor GCN4, where a short C-terminal stretch of the protein forms a highly stable dimeric coiled-coil structure, termed a leucine zipper.

Accordingly, in a first aspect, provided herein is the use of an oligomeric protein as a binding agent for binding to lipopolysaccharide (LPS), the oligomeric protein having a coiled-coil structure comprising at least 2 monomer peptides, wherein each monomer peptide, which may be the same or different, is capable of forming an α-helix and comprises at least one core sequence having at least 60% sequence identity to the heptad repeat sequence of SEQ ID NO. 1.

In keeping with the characteristic feature of coiled coil proteins which comprise, or are made up of, amphipathic α-helices (or α-helical strands), the oligomeric coiled coil protein has a hydrophobic core. The hydrophobic core comprises hydrophobic residues which face each other in the hydrophobic core structure.

Thus, in particular, the core sequence of a peptide monomer may comprise at least 3 heptad motifs a-b-c-d-e-f-g, or variants thereof, each variant comprising no more than 1 insertion or deletion to the heptad motif. Further, in an embodiment at least 50% of the amino acid residues corresponding to positions a and d of the heptad motifs or variants thereof are hydrophobic residues. In another embodiment, at least 75% of the amino acid residues corresponding to positions a and d of the heptad motifs or variants thereof are hydrophobic residues

Accordingly, in one embodiment of this aspect, provided herein is the use of an oligomeric protein as a binding agent for binding to lipopolysaccharide (LPS), the oligomeric protein having a coiled-coil structure comprising at least 2 monomer peptides, wherein each monomer peptide, which may be the same or different, is capable of forming an α-helix and comprises at least one core sequence having at least 60% sequence identity to the heptad repeat sequence of SEQ ID NO. 1, wherein the core sequence comprises at least 3 heptad motifs a-b-c-d-e-f-g, or variants thereof, each variant comprising no more than 1 insertion or deletion to the heptad motif, wherein at least 50% of the amino acid residues corresponding to positions a and d of the heptad motifs or variants thereof are hydrophobic residues.

The composition of the amino acid residues of the hydrophobic core of a coiled core protein need not be entirely, or solely, of hydrophobic residues, and other residues can be present, including hydrophilic, e.g. polar, residues. Thus, in certain embodiments, at least 52.5, 55, 60, 62.5, 70, or 75% of the amino acid residues corresponding to positions a and d of the heptad motifs or variants thereof are hydrophobic residues.

In a second aspect, provided herein is a method of binding LPS, the method comprising contacting the LPS, or a sample containing LPS, with an oligomeric protein as defined herein, to allow the protein to bind to the LPS to form a protein-lipopolysaccharide complex.

In an embodiment, the method is an in vitro method.

In a third aspect, provided herein is a kit for use as a binding agent for binding to LPS as defined herein or for use in the method of binding LPS as defined herein, said kit comprising:

(i) an oligomeric protein as defined herein; and

(ii) at least one non-denaturing detergent.

The use and method herein may be for use in detecting and/or removing LPS in or from a sample.

Due to its ability to bind to a wide range of endotoxins with high affinity, the present oligomeric protein is suitable for use in a variety of applications involving endotoxin binding, detection and removal. In this regard, the present oligomeric protein may be immobilised on a solid substrate. The oligomeric protein may, for example, be immobilised on a resin for use in a column or filter, e.g. a spin column filter or a flow column, to bind to and remove endotoxins from a sample applied to said filter or column, as in endotoxin removal methods outlined above. The oligomeric protein may also be used in endotoxin detection systems, both to detect the presence of endotoxins in a given sample, and also to certify samples, reagents, products, etc. as being endotoxin-free, where endotoxins are not detected. In this regard, the oligomeric protein may be provided in the form of a conjugate or a fusion with a second component, such as a conjugate with a detection moiety, or a fusion protein with a suitable fusion partner, to facilitate the detection of endotoxins.

In a fourth aspect, provided herein is a product comprising an oligomeric protein as defined herein immobilised on a solid substrate.

As noted above, it is understood that the oligomeric protein as defined herein interacts with LPS via the lipid A component. Thus, in a fifth aspect, provided herein is the use of an oligomeric protein as defined herein as a binding agent for binding to lipid A of LPS.

Similarly, provided herein is, in a sixth aspect, a method of binding lipid A of LPS, the method comprising contacting the lipid A, or a sample containing lipid A, with an oligomeric protein as defined herein, to allow the protein to bind to the lipid A to form a protein-lipopolysaccharide complex.

In a seventh aspect, provided herein is a kit for use as a binding agent for binding to lipid A as defined herein or for use in the method of binding lipid A as defined herein, said kit comprising:

(i) an oligomeric protein as defined herein; and

(ii) at least one non-denaturing detergent.

The oligomeric protein described herein provides an alternative binding agent for binding to LPS. In an embodiment, the disclosure herein provides an improved binding agent for LPS.

The LPS binding agent herein has an number of advantages. Further it can be seen to address a number of the problems outlined above that are associated with known LPS binding and detection methods.

In terms of LPS detection, the oligomeric protein described herein removes the need to use an expensive lysate harvested from horseshoe crabs, and avoids the problems of consistency and reproducibility which are associated with the use of LPS detection methods that rely on the use of Factor C, such as the LAL assay, and recombinant variants thereof.

In addition, the oligomeric protein described herein is capable of dissolving LPS aggregates. Accordingly, the oligomeric protein can mitigate LPS masking and effectively increase the measurable concentration of LPS in a sample comprising such LPS aggregates. This allows for low concentrations of LPS in a sample to be detected.

The oligomeric protein described herein comprises a relatively short peptide sequence, and thus in some embodiments it may be produced synthetically, without the need for any biological expression systems.

### DESCRIPTION

The oligomeric protein disclosed herein has an oligomeric alpha-helical coiled-coil structure. Coiled-coils are ubiquitous protein elements consisting of two or more amphipathic α-helices wound into supercoiled bundles (Lupas and Gruber, 2005). A key characteristic of amphipathic alpha-helical coiled-coils is the repeating heptad motif a-b-c-d-e-f-g where positions a and d are predominantly occupied by hydrophobic residues, and positions b, c, e, f and g are predominantly occupied by hydrophilic residues. Alpha-helices comprise 3.6 residues per turn, which means that the repeating heptad motif places the residues in positions a and d on the same face of the helical structure. This facilitates the formation of highly stable supercoils with the hydrophobic residues facing in towards each other in what is termed the hydrophobic core, whilst the hydrophilic residues face outwards. It will be noted that whilst the hydrophobic core of a coiled coil protein typically comprises predominantly hydrophobic residues, it is not necessary for all of the residues in the core structure to be hydrophobic, and coiled coil proteins are known which may comprise other residues located in the core structure, e.g. polar residues, which nonetheless are able to retain a coiled coil structure.

The oligomeric protein herein is based on a variant of the leucine zipper sequence of the protein GCN4, known asGCN4-pIL, where GCN4-p‘ad’ refers to the amino acids which are present at positions a and d in the heptad motif. It has been demonstrated that by mutating the hydrophobic core residues present at positions a and d, in particular by varying the ratio of leucine and isoleucine residues present at these positions, it is possible to alter the preferred oligomeric state of the protein structure from dimers to trimers (GCN4-pII) and tetramers (GCN4-pLI) (Harbury et al., 1993; Delano and Brunger, 1994).

The stability of these coiled-coil elements, and their propensity to form oligomers has led to the use of GCN4 coiled-coil structures as chimeric extensions (i.e. fusion partners) to induce oligomerisation and to stabilize oligomeric structures in fusion proteins, as well as to increase the solubility of such proteins. In this regard, the present inventors were initially intending to investigate a putative interaction between LPS and two domains belonging to the trimeric autotransporter adhesin, SadA. In order to study this protein, two SadA constructs were prepared, K9 and K14 (see Example 1 below), both of which were stabilized by flanking GCN4-pII segments. It was surprisingly found, however, that the GCN4-pII adapters which were being used to stabilize the SadA constructs displayed an extremely high affinity for LPS, with a K_{D} in the nanomolar range.

Following this serendipitous finding, the present inventors have developed an oligomeric protein having a coiled-coil structure based on the GCN4-pII protein which is capable of being used as a binding agent for binding to LPS. Further experimentation has revealed that that the interaction between this protein and LPS occurs via binding of the protein to the lipid A component of LPS. As noted above, the structure of the lipid A component is highly conserved among Gram-negative bacterial species, and thus the present oligomeric protein is understood to be capable of binding to a wide range of bacterial endotoxins, with extremely high affinity. Moreover, the present oligomeric protein can be recombinantly overexpressed in typical expression systems and can be purified from inclusion bodies without interacting with any naturally occurring endotoxins, which allows for large-scale, sustainable and cost-effective production.

The oligomeric protein described herein comprises at least 2 monomer peptides. These monomer peptides represent the individual subunits that, as a whole, make up the oligomeric protein. Each monomer is capable of forming an alpha-helix. The monomers may be provided as separate peptides in the sense of separate peptide chains, or strands, which interact together to form the oligomeric protein. In such an embodiment, the peptide monomers may thus be regarded as individual sub-units of the protein, that is, separate monomer peptide units. Thus in some embodiments each alpha-helix in the oligomeric protein may be considered to correspond to a separate monomer.

In other embodiments, the monomer peptides may be linked together. Thus, the monomer peptides may be linked, or connected, by linker sequences. In such an embodiment the oligomeric protein has a single-chain format in terms of its primary structure or sequence, although of course the monomer peptides interact to form an oligomeric coiled coil structure which can be seen to have “strands” which interact to form the coiled coil structure. In such an embodiment the monomer peptides may be regarded as domains of the single-chain protein sequence. More particularly, the oligomeric protein may be seen to have a 3D structure made up of the monomer peptide domains.

Each monomer peptide comprises at least one core sequence, which has at least 60% sequence identity to the heptad repeat sequence of SEQ ID NO: 1. SEQ ID NO: 1 represents a variant of the sequence of the model peptide GCN4-pII, which is based on the sequence from the dimerization motif in the C-terminal of the GCN4 protein, and which comprises the repeating heptad motif a-b-c-d-e-f-g, with isoleucine residues present at positions a and d in the motif, as shown below. In some embodiments, the core sequence may have at least 65%, 70%, 75%, 80%, 85%, 90%, 95%, 97%, 98%, or 99% sequence identity to SEQ ID NO: 1. In some embodiments the core sequence may comprise or consist of the sequence of SEQ ID NO: 1.

Sequence identity may be determined by any suitable means known in the art, e.g. using the SWISS-PROT protein sequence databank using FASTA pep-cmp with a variable pamfactor, and gap creation penalty set at 12.0 and gap extension penalty set at 4.0, and a window of 2 amino acids. Other programs for determining amino acid sequence identity include the BestFit program of the Genetics Computer Group (GCG) Version 10 Software package from the University of Wisconsin. The program uses the local homology algorithm of Smith and Waterman with the default values: Gap creation penalty—8, Gap extension penalty=2, Average match=2.912, Average mismatch=−2.003. In one embodiment said comparison is made over the full length of the core sequence.

SEQ ID NO. 1:
M K Q I E D K I E E I L S K I Y H I E N E I A R I K K L I K
d e f g a b c d e f g a b c d e f g a b c d e f g a

It can be seen that SEQ ID NO: 1 comprises multiple repeats of the a-b-c-d-e-f-g heptad motif. As depicted above, the a and d residues are I. However, as will be discussed in more detail below, they may be varied, and in one embodiment they may be I or L, or derivatives thereof, or other hydrophobic residues. As noted above, not all a and d residues in a heptad motif need be hydrophobic. It suffices that there are enough hydrophobic residues for an oligomeric coiled coil structure to form. This may depend on sequence context, and the other residues that are present in the sequence of the monomer peptides.

In an embodiment, the core sequence of each monomer peptide comprises at least 3 heptad motifs a-b-c-d-e-f-g, or variants thereof. Although the heptad motif is conventionally written as a-b-c-d-e-f-g, there is no requirement in practice that the heptad repeat sequence in the core sequence begins with position a. The motif repeats, with position a following position g and thus the heptad motif can begin at any position, provided that it comprises all 7 positions a-b-c-d-e-f-g in consecutive order. Accordingly, the motif d-e-f-g-a-b-c is a valid heptad motif, for example. In some embodiments, the core sequence may comprise one or more variants of the heptad motif a-b-c-d-e-f-g, wherein each variant comprises no more than one insertion or deletion to the heptad motif. These variants of the heptad motif a-b-c-d-e-f-g comprising an insertion or a deletion are collectively referred to as “variant motifs”. In this context, the terms “insertion” and “deletion” refer to the addition of a single residue to the heptad motif, and the removal of a single residue from the heptad motif, respectively.

The insertion or deletion may be made at any position within the heptad motif, including at either end of the heptad motif. For example, considering an insertion of residue X into the motif a-b-c-d-e-f-g, the resulting motif may be X-a-b-c-d-e-f-g, a-X-b-c-d-e-f-g, a-b-X-c-d-e-f-g, etc. Importantly, it can be seen that the labelling of the remaining positions within the motif remains unchanged. This applies in the case of both insertions and deletions. Thus, if the residue at position b is deleted, for example, the remaining motif would comprise the sequence a-c-d-e-f-g.

The insertion or deletion of multiple consecutive residues is not considered to be one insertion or deletion. Accordingly, the at least three heptad motifs or variant motifs present in each core sequence must not contain more insertions and deletions to the heptad motifs a-b-c-d-e-f-g than the total number of variant motifs which are present. Insertions or deletions which are adjacent to each other in the core sequence are only permissible if they are at adjacent ends of consecutive variant motifs, and if the consecutive variant motifs each comprise only one insertion or deletion. In such a case, the adjacent insertions/deletions can be seen to be the product of two separate insertions/deletions in two separate variant motifs.

In some embodiments, the core sequence comprises at least 4 heptad or variant motifs. In some embodiments, the core sequence comprises 3 to 5 heptad or variant motifs. For example, the core sequence may comprise 3, 4, or 5 heptad or variant motifs. In some embodiments, the core sequence may comprise at least 3 heptad motifs, and no variant motifs. In other embodiments, the core sequence may comprise at least 3 variant motifs, and no heptad motifs. Moreover, the core sequence may comprise any combination of at least 3 heptad motifs and variant motifs, and these heptad and variant motifs may be arranged in any order.

As discussed above, coiled-coil protein structures depend upon the coordinated arrangement of hydrophobic residues within the repeating heptad motif present in each alpha helix. The hydrophobic residues within each alpha helix are positioned such that they are predominantly presented on a single face of that alpha helix. Alternatively put, the residues within each alpha helix are arranged such that the residues presented on one face of that alpha helix are predominantly hydrophobic. This allows the hydrophobic faces of each alpha helix in the oligomeric protein to form a stable hydrophobic core in the centre of the protein structure. It is not essential that hydrophobic residues are present at both position a and position d within every repeat of the heptad motif, but typically the majority of these positions are occupied by hydrophobic residues. To facilitate this structure in the oligomeric coiled-coil protein defined herein, in an embodiment, within the core sequence, at least 50% of the amino acid residues corresponding to positions a and d of the heptad motifs or variants thereof are hydrophobic residues. As shown in the schematic above, positions a and d of the heptad motif in SEQ ID NO: 1 are represented by positions 4, 8, 11 15, 18, 22, 25, and 29 of the sequence. Thus it can be seen that, as an alternative definition, at least 50% of the amino acid residues at positions corresponding to positions 4, 8, 11 15, 18, 22, 25, and 29 of SEQ ID NO.1 are hydrophobic residues. Thus, in an embodiment at least 4 out of 8 “a” or “d” positions in the heptad repeat sequence of SEQ ID NO.1 are hydrophobic residues.

More particularly, at least 52.5%. 55%, 60%. 62.5%, or 70% of the amino acid residues corresponding to positions a and d of the heptad motifs in the heptad repeat sequence of SEQ ID NO. 1 are hydrophobic. However, in one representative embodiment, at least 75% of the amino acid residues corresponding to positions a and d of the heptad motifs in the heptad repeat sequence of SEQ ID NO. 1 are hydrophobic. Thus, in an embodiment at least 6 out of 8 “a” or “d” positions in the heptad repeat sequence of SEQ ID NO.1 are hydrophobic residues.

By way of representative example, in the sequence of SEQ ID NO. 1, at least 4, 5, 6 or 7 of the a or d residues, or the positions corresponding to positions 4, 8, 11 15, 18, 22, 25, and 29 of SEQ ID NO.1 may be hydrophobic residues.

Based on knowledge of coiled coil protein structures and sequences, it would be within the routine skill of the person skilled in the art to make sequence modifications, including substitution of the residues at positions a and d, relative to other positions in the heptad motifs, to obtain coiled coil structures based on modified or variant peptides of SEQ ID NO. 1.

The term “hydrophobic residues” as used herein includes the residues of any amino acid recognised or identified in the art as being hydrophobic. Such amino acids include the following proteogenic amino acids: leucine, isoleucine, valine, alanine, methionine, phenylalanine, proline and glycine. However, in an embodiment the hydrophobic residues are selected from the amino acids leucine, isoleucine, valine, alanine, methionine, phenylalanine, or chemical derivatives thereof. In another embodiment the hydrophobic residues are selected from: leucine, isoleucine, valine, alanine, and methionine, and chemical derivatives of these amino acids. The hydrophobic residues present in the core sequence may also include non-conventional hydrophobic amino acids, i.e. hydrophobic amino acids which possess a side chain that is not coded for by the standard genetic code. In particular, fluoro-derivatives of these amino acids, such as fluoroisoleucine and fluoroleucine are included. Other known derivatives include seleno-derivatives, e.g. selenomethionine. Further examples of such non-conventional hydrophobic amino acids, including D-amino acid variants (where D-amino acids are included, all the amino acids may be D-amino acids), L-N methylamino acid variants, D-α methylamino acid variants and D-N-methylamino acid variants of the conventional hydrophobic amino acids defined above are listed in Table 1 below.

TABLE 1

| Non-conventional | | Non-conventional | |
|---|---|---|---|
| amino acid | Code | amino acid | Code |
| α-aminobutyric acid | Abu | L-N-methylalanine | Nmala |
| α-amino-α-methylb- | Mgabu | L-N-methylisolleucine | Nmile |
| utyrate | | L-N-methylleucine | Nmleu |
| aminocyclopropane- | Cpro | L-N-methylvaline | Nmval |
| carboxylate | | L-N-methylethylglycine | Nmetg |
| aminoisobutyric acid | Aib | L-N-methylmethionine | Nmmet |
| aminonorbornyl- | Norb | L-N-methylnorvaline | Nmnva |
| carboxylate | | L-N-methylnorleucine | Nmnle |
| α-methyl-γ-amino- | Mgabu | L-N-methyl-t-butylglycine | Nmtbug |
| butyrate | | penicillamine | Pen |
| γ-aminobutyhc acid | Gabu | α-methylcyclohexylalanine | Mchexa |
| cyclohexylalanine | Chexa | α-methyl-aminoisobutyrate | Maib |
| cyclopentylalanine | Cpen | α-napthylalanine | Anap |
| D-alanine | Dal | α-methylcylcopentyl- | Mcpen |
| D-valine | Dval | alanine | |
| D-leucine | Dleu | α-methyl-α-napthylalanine | Manap |
| D-isoleucine | Dile | α-methylpenicillamine | Mpen |
| D-methionine | Dmet | L-norleucine | Nle |
| N-cyclopropylglycine | Nepro | L-norvaline | Nva |
| N-cyclobutylglycine | Ncbut | L-methylethylglycine | Metg |
| N-cyclohexylglycine | Nchex | L-t-butylglycine | Tbug |
| N-cycloheptylglycine | Nchep | L-ethylglycine | Etg |
| N-cyclooctylglycine | Ncoct | L-α-methylnorleucine | Mnle |
| N-cyclodecylglycine | Ncdec | L-α-methylalanine | Mala |
| N-cycloundecylglycine | Ncund | L-α-methyl-t-butylglycine | Mtbug |
| N-cylcododecylglycine | Ncdod | L-α-methylisoleucine | Mile |
| N-(2,2-diphenylethyl) | Nbhm | L-α-methylleucine | Mleu |
| glycine | | L-α-methylmethionine | Mmet |
| N-methylpenicillamine | Nmpen | L-α-methylnorvaline | Mnva |
| N-methylcyclohexyl- | Nmchexa | L-α-methylvaline | Mval |
| alanine | | N-(1-methylethyl)glycine | Nval |
| N-methylcyclopentyl- | Nmcpen | | |
| alanine | | | |
| N-methylglycine | Nala | | |
| N-(1-methylpropyl) | Nile | | |
| glycine | | | |

| | |
|---|---|
| N-(2-methylpropyl) | Nleu |
| glycine | |
| N-(3,3-diphenylpropyl) | Nbhe |
| glycine | |
| N-(2-methylthioethyl) | Nmet |
| glycine | |
| D-α-methylalanine | Dmala |
| D-α-methylisoleucine | Dmile |
| D-α-methylleucine | Dmleu |
| D-α-methylmethionine | Dmmet |
| D-N-methylalanine | Dnmala |

| | |
|---|---|
| D-N-methylisoleucine | Dnmile |
| D-N-methylleucine | Dnmleu |
| D-N-methylmethionine | Dnmmet |
| D-N-methylvaline | Dnmval |
| N-benzylglycine | Nphe |
| N-methyla-napthyl- | Nmanap |
| alanine | |
| N-methylaminoiso- | Nmaib |
| butyrate | |

In some embodiments, at least 80%, 85%, 90%, 95%, 97%, 98% or 99% of the amino acid residues corresponding to positions a and d of the heptad motifs or variants thereof are hydrophobic residues. Alternatively expressed, at least 80%, 85%, 90%, 95%, 97%, 98% or 99% of the amino acid residues at positions corresponding to positions 4, 8, 11 15, 18, 22, 25, and 29 of SEQ ID NO.1 are hydrophobic residues.

In some embodiments, 100% of the amino acid residues corresponding to positions a and d of the heptad motifs or variants thereof are hydrophobic residues. Alternatively expressed, 100% of the amino acid residues at positions corresponding to positions 4, 8, 11 15, 18, 22, 25, and 29 of SEQ ID NO.1 are hydrophobic residues.

The hydrophobic residues within the core sequence may all be the same, or they may be different to each other. In some embodiments, each hydrophobic residue in the heptad or variant motifs is independently selected from the group consisting of leucine, isoleucine, valine, alanine, methionine, and chemical derivatives thereof, including fluoro-derivatives thereof. In one embodiment, each hydrophobic residue in the heptad or variant motifs is independently selected from the group consisting of leucine, isoleucine, valine, methionine, and chemical derivatives thereof, including fluoro-derivatives or seleno-derivatives thereof. In one embodiment, each hydrophobic residue in the heptad or variant motifs is independently selected from the group consisting of leucine, isoleucine and chemical derivatives thereof, e.g. fluoroleucine and fluoroisoleucine.

In some embodiments, at least 50% of the hydrophobic residues in the heptad or variant motifs are isoleucine or fluoroisoleucine. In some embodiments, at least 60%, 70%, 75%, 80%, 85%, 90%, 95%, 97%, 98%, or 99% of the hydrophobic residues in the heptad or variant motifs are isoleucine or fluoroisoleucine. In some embodiments, 100% of the hydrophobic residues in the heptad or variant motifs are isoleucine or fluoroisoleucine.

The residues which do not form part of the hydrophobic core of the coiled-coil structure, i.e. the residues at positions b, c, e, f and g, are generally closer to the surface of the protein, and are thus exposed to the environment. The identity of these residues is not critical and they can be varied. In some embodiments, at least 50% of the amino acid residues corresponding to positions b, c, e, f and g are polar residues. In some embodiments, at least 60%, 70%, 75%, 80%, 85%, 90%, 95%, 97%, 98%, or 99% of the amino acid residues corresponding to positions b, c, e, f and g are polar residues. In some embodiments, 100% of the amino acid residues corresponding to positions b, c, e, f and g are polar residues.

The term “polar residues” as used herein includes the residue of any amino acid recognised or identified in the art as polar. This includes charged amino acids. A polar amino acid residue may be selected from the residues of the amino acids serine, threonine, asparagine, glutamine, aspartic acid, glutamic acid, histidine, arginine, lysine, tyrosine, cysteine, tryptophan, methionine, and chemical derivatives of these amino acids. In one embodiment, a polar amino acid residue may be selected from the residues of the amino acids serine, threonine, asparagine, glutamine, aspartic acid, glutamic acid, histidine, arginine, lysine, tyrosine. In addition, the polar residues present in the core sequence may also include non-conventional polar amino acids, i.e. polar amino acids which possess a side chain that is not coded for by the standard genetic code. Examples of such non-conventional polar amino acids, including D amino acid variants, amide isostere variants (such as N-methyl amide, retro-inverse amide, thioamide, thioester, phosphonate, ketomethylene, hydroxymethylene, fluorovinyl, (E)-vinyl, methyleneamino, methylenethio or alkane), L-N methylamino acid variants, D-α methylamino acid variants and D-N-methylamino acid variants of the conventional polar amino acids defined above are listed in Table 2 below. As noted above, where D-amino acids are used, all the amino acids in the monomer peptides may be D-amino acids.

TABLE 2

| Non-conventional | | Non-conventional | |
|---|---|---|---|
| amino acid | Code | amino acid | Code |
| L-N-methylarginine | Nmarg | D-α-methylthreonine | Dmthr |
| N-(4-aminobutyl)glycine | Nglu | D-α-methylserine | Dmser |
| L-N-methylasparagine | Nmasn | D-α-methyltyrosine | Dmty |
| L-N-methylaspartic acid | Nmasp | N-(2-aminoethyl)glycine | Naeg |
| L-N-methylglutamine | Nmgln | N-(3-aminopropyl)glycine | Norn |
| L-N-methylglutamic acid | Nmglu | N-amino-α-methylbutyrate | Nmaabu |
| L-N-methylhistidine | Nmhis | N-(imidazolylethyl)) | Nhis |
| L-N-methyllysine | Nmlys | glycine | |
| L-N-methylornithine | Nmorn | N-(p-hydroxyphenyl) | Nhtyr |
| L-N-methylthreonine | Nmthr | glycine | |
| L-N-methyltyrosine | Nmtyr | N-(3-guanidinopropyl) | Narg |
| L-N-methylserine | Nmser | glycine | |
| D-arginine | Darg | N-(1-hydroxyethyl)glycine | Nthr |
| D-aspartic acid | Dasp | N-(hydroxyethyl))glycine | Nser |
| D-glutamine | Dgln | N-(2-carboxyethyl)glycine | Nglu |
| D-glutamic acid | Dglu | D-N-methylarginine | Dnmarg |
| D-histidine | Dhis | D-N-methylasparagine | Dnmasn |
| D-lysine | Dlys | D-N-methylaspartate | Dnmasp |
| D-ornithine | Dorn | D-N-methylglutamine | Dnmgln |
| D-serine | Dser | D-N-methylglutamate | Dnmglu |
| D-threonine | Dthr | D-N-methylhistidine | Dnmhis |
| D-tyrosine | Dtyr | D-N-methyllysine | Dnmlys |
| D-α-methylarginine | Dmarg | D-N-methylornithine | Dnmorn |
| D-α-methylasparagine | Dmasn | D-N-methylserine | Dnmser |
| D-α-methylaspartate | Dmasp | D-N-methyltyrosine | Dnmtyr |
| D-α-methylglutamine | Dmgln | D-N-methylthreonine | Dnmthr |
| D-α-methylhistidine | Dmhis | L-α-methylarginine | Marg |
| D-α-methyllysine | Dmlys | L-α-methylasparagine | Masn |
| D-α-methylornithine | Dmorn | L-α-methylglutamate | Mglu |
| L-α-methylserine | Mser | L-α-methyllysine | Miys |
| L-α-methylaspartate | Masp | L-α-methylornithine | Morn |
| L-α-methylglutamine | Mgin | L-α-methylthreonine | Mthr |
| L-α-methylhistidine | Mhis | | |
| L-α-methyltyrosine | Mtyr | | |
| N-(N-(2,2-diphenylethyl) | Nnbhm | | |
| carbamylmethyl)glycine | | | |
| N-(N-(3,3-diphenyl- | Nnbhe | | |

| | |
|---|---|
| propyl) | |
| carbamylmethyl)glycine | |
| 1-carboxy-1-(2,2- | Nmbc |
| diphenyl- | |
| ethylamino)cyclopropane | |
| L-O-methyl serine | Omser |
| L-O-methyl homoserine | Omhse |

Whilst the consistent arrangement of hydrophobic and polar amino acids within the heptad motif is responsible for the structure of coiled-coil proteins, the general rules regarding the location of the residues are not immutable. Thus, just as not every residue corresponding to position a or d within the heptad or variant motifs of the core sequence must be hydrophobic, similarly, not every residue corresponding to positions b, c, e, f or g within the heptad or variant motifs of the core sequence must be polar. In some embodiments, at least 5% of the amino acid residues corresponding to positions b, c, e, f and g may be aliphatic residues. In some embodiments, at least 10% or at least 15% of the amino acid residues corresponding to positions b, c, e, f and g may be aliphatic residues.

The term “aliphatic residues” as used herein includes the amino acids glycine, alanine, isoleucine, leucine, proline, valine and methionine, and chemical derivatives of these amino acids, in particular fluoro-derivatives thereof, including fluoroleucine and fluoroisoleucine. In addition, the aliphatic residues present in the core sequence may also include non-conventional aliphatic amino acids, i.e. aliphatic amino acids which possess a side chain that is not coded for by the standard genetic code, such as D amino acid variants, and other non-conventional aliphatic amino acids.

The core sequence may comprise a specific percentage of polar residues, as defined above, and a specific percentage of aliphatic residues, as defined above. For example, in some embodiments, at least 50% (or higher, as defined above) of the amino acid residues corresponding to positions b, c, e, f and g may be polar residues, and at least 5% (or higher, as defined above) of amino acid residues corresponding to positions b, c, e, f and g may be aliphatic residues polar residues. However, this is not essential, and as noted above, can be varied.

In some embodiments, the core sequence, as defined herein, may be flanked on one or both sides by a flanking amino acid sequence. If the core sequence is flanked on both sides, the flanking sequence on one side of the core sequence may be the same as or different to the flanking sequence on the other side of the core sequence. The flanking sequence may or may not form part of the coiled coil structure of the oligomeric protein. Thus, the flanking sequence may contribute to, or be part of, the α-helical structure of a monomer peptide and/or may otherwise contribute to or form part of the coiled coil structure, or it may be a separate part of the monomer peptide sequence. A flanking sequence may be used to perform various functions, or to impart a property to the oligomeric protein. For example it may be used to extend the heptad repeat sequence of a monomer peptide, to assist in oligomerisation of the monomer peptides, to link the monomer peptides (e.g. within a single-chain construct), or to provide a separate functional moiety to the oligomeric protein.

The length of a flanking sequence is not critical and it may be varied according to need and desire, or the nature of the flanking sequence and/or its purpose. It may for example be from 1 to 300 amino acids, for example from any one of 2, 3, 4, 5, 6, or 7 to any one of 270, 250, 240, 230, 220, 210 or 200 amino acids. These ranges are given for example only, and there is no restriction on the length of the flanking sequence. In some embodiments in practice the flanking sequence may be up to 170, 160, 150, 140, 130, 120, 110, 100, 90, 80, 70, 60, 50, 40, 30, 20, or 10 amino acids. In some embodiments a short flanking sequence is preferred e.g. up to 20, 15, 12, 10, 8, 7 or 6.

Accordingly in some embodiments, the flanking sequence may comprise one or more heptad motifs, and/or one or more parts thereof. In this context, a part of a heptad motif may include 1, 2, 3, 4 or 5 residues which make up a consecutive portion of a heptad motif. In some embodiments, the heptad motif in the flanking sequence corresponds to a heptad motif as found in SEQ ID NO. 1, or in a sequence having at least 60% (e.g. at least 70%, 80%, 90%) sequence identity thereto, with the proviso that at least one of the amino acid residues corresponding to positions a and d of the heptad motif is a hydrophobic residue. In some embodiments, the flanking sequence may comprise SEQ ID NO. 1, or a part thereof, or a sequence having at least 60% (e.g. at least 70%, 80%, 90%) sequence identity thereto. Further, in such an embodiment at least 50% (e.g. at least 75%) of the amino acid residues corresponding to positions a and d of the heptad motifs of SEQ ID NO. 1, or variants thereof, are hydrophobic residues.

When the flanking sequence comprises one or more heptad motifs, or one or more parts thereof, it may be seen as a continuation of the heptad motifs of the core sequence. As a result, the alpha-helix of the monomer protein which forms part of the coiled-coil structure of the oligomeric protein may be extended beyond the end of the core sequence.

In some embodiments, the heptad motifs of the core sequence and the heptad motifs of the flanking sequence are continuous. That is to say, that the first residue of the flanking sequence (i.e. the residue immediately adjacent to the end of the core sequence) corresponds to the position of the heptad motif which follows the position corresponding to the adjacent terminal residue of the core sequence. In this manner, the repeating heptad motif a-b-c-d-e-f-g is preserved with no gap between the heptad motifs of the core sequence and the heptad motifs of the flanking sequence.

In other embodiments the flanking sequence may comprise one or more heptad motifs which are not entirely continuous with the heptad motifs of the core sequence, i.e. there may be one or more residues between the heptad repeats in the core sequence and the heptad repeats in the flanking sequence which do not form part of a continuous repeating heptad motif.

In some embodiments, the core sequence and the flanking sequence may be arranged such that each monomer peptide does not comprise more than 8 repeats of the heptad motif. In some embodiments, the monomer peptide does not comprise more than 7 repeats, more than 6 repeats, or more than 5 repeats of the heptad motif. In other words, a monomer peptide of the oligomeric protein may comprise up to 8, 7, 6 or 5 heptad repeats.

In some embodiments, the flanking sequence of the monomer peptide may not entirely form a continuous alpha-helix with the core sequence, and thus may not entirely be part of the coiled-coil structure of the oligomeric peptide.

In some embodiments the oligomeric protein defined herein may be in the form of a conjugate or a fusion with one or more additional components or moieties. As will be set out in more detail below, the oligomeric protein may be in the form of a conjugate with a detection moiety, an oligomerisation moiety, or an immobilising moiety, or indeed any desired component or moiety, e.g. a functional or structural component or moiety. The conjugated moiety may be of any chemical or physical nature, e.g. a small molecule or a macromolecule. The oligomeric protein may be in the form of a fusion protein with a fusion partner. Thus, a detection or immobilisation, or other additional moiety may be proteinaceous in nature, i.e. it may or may not be a polypeptide component (the term “polypeptide” is used herein to include any peptide, polypeptide or protein, regardless of length). An oligomerisation moiety may be a polypeptide. In addition, the oligomeric protein may be immobilised on a solid substrate. Accordingly, in some embodiments, the one or more additional components may be a detection moiety, an oligomerisation moiety, an immobilising moiety or a fusion partner.

In some embodiments, the one or more additional components with which the oligomeric protein is conjugated or fused may form all or part of a flanking sequence within one or more of the monomer peptides which make up the oligomeric protein. In other embodiments, the conjugated moiety may be a separate component (i.e. separate to the oligomeric protein, or a monomer peptide thereof).

It will be understood that the presence of an additional component within a flanking sequence may be in addition to, or as an alternative to the presence of one or more heptad motifs in the same flanking sequence. That is to say, a given flanking sequence may comprise both one or more heptad motifs, or parts thereof, and one or more additional components. Where a flanking sequence does comprise one or more heptad motifs, or parts thereof, and one or more additional components, the flanking sequence may be arranged such that the one or more heptad motifs, or parts thereof, are closer to the core sequence than the one or more additional components.

In some embodiments, the oligomeric protein is in the form of a fusion or a conjugate with a single additional component. The additional component may form all or part of a flanking sequence within one of the monomer peptides. Alternatively, the oligomeric protein may be in the form of a fusion or a conjugate with 2 or more additional components. In some embodiments, the additional components may form all or part of the same flanking sequence within the same monomer peptide. In some embodiments, a single monomer peptide may comprise a core sequence flanked on both sides by flanking sequences, wherein each flanking sequence comprises one or more additional components. Additionally, an oligomeric protein as defined herein may comprise multiple monomer peptides which each comprise one or more additional components, in any of the arrangements set out above.

In the case of an oligomeric protein in the form of a conjugate with an oligomerisation moiety, the oligomerisation moiety may be made up of several oligomerisation sequences, wherein each monomer peptide comprises an oligomerisation sequence. Accordingly, the oligomeric protein may be comprised of at least 2 monomer peptides, wherein each monomer peptide comprises an oligomerisation sequence in a flanking sequence.

The coiled-coil structure of the oligomeric protein disclosed herein may form spontaneously when the monomer peptides are brought into contact with each other. Alternatively, the formation of the oligomeric structure may require a ‘trigger’ to overcome kinetic hindrances and to bring the monomer peptides together. Moreover, in some embodiments, it may be necessary to stabilise the oligomeric coiled-coil structure of the protein. This initiation and stabilisation of the oligomeric coiled-coil structure may be achieved by an oligomerisation sequence. An oligomerisation sequence is a protein sequence which is capable of oligomerising, i.e. interacting with other copies of itself so as to form oligomers. It will be understood that oligomerisation is cooperative, which is to say that where a particular portion of a larger protein is capable of readily and stably oligomerising, this can help to induce oligomerisation in the remainder of the protein structure, where it would not otherwise occur. For example, the head domains of adhesion proteins, such as the YadA head domain, are known in the art to be capable of inducing the formation of coiled coil structures that otherwise would not be stable enough to form. GCN4 proteins have also been used in a similar manner to stabilise trimeric autotransporter adhesins (Hartmann et al, 2012). This domain, or other equivalent domains known in the art, may thus be used as an oligomeric sequence. As noted above, it may be that when the oligomeric protein is conjugated with an oligomerisation moiety, each monomer peptide within the oligomeric protein comprises an oligomerisation sequence.

Additionally or alternatively, in some embodiments, the initiation and stabilisation of the coiled-coil structure may be done by linking the monomer peptides together.

Although the monomer peptides defined herein can to some degree be considered in isolation, in some embodiments, 2 or more of the monomer peptides within the oligomeric protein disclosed herein may be linked together. Accordingly, the flanking sequence may comprise one or more linker sequences. This may be in addition to, or as an alternative to, the one or more heptad motifs or parts thereof, and the one or more additional components which may be contained in a flanking sequence. It will be understood that the flanking sequence may comprise any combination of heptad motifs and/or parts thereof, one or more additional components, and/or one or more linker sequences.

The linker sequences are capable of linking one monomer peptide to another monomer peptide, so as to form a single peptide chain within at least a portion of the oligomeric protein. Where two monomer peptides are linked together via a linking sequence, it may be considered that one of the monomer peptides (i.e. the first monomer peptide) comprises a flanking sequence containing the entire linker sequence, which joins directly to the core sequence of the other monomer peptide (i.e. without the second monomer peptide having a flanking sequence at that end of the core sequence). Alternatively, the link between the two monomer peptides may be considered to be made up partly of a flanking sequence of the first monomer peptide, and partly of a flanking sequence of the second monomer peptide (i.e. wherein both monomer peptides comprise a flanking sequence comprising a linker sequence).

Where a linker sequence is included in a monomer peptide to link the monomer peptides together, it may be convenient for the flanking sequence between two monomer peptides not to contain anything other the linker sequence and optionally heptad repeat motifs or parts thereof. However, a flanking sequence at either end of a chain of linked monomer peptides may comprise an additional sequence (e.g. as discussed above). Alternatively expressed, in such a linked, e.g. single chain construct, the oligomeric protein may be in the form of a conjugate with an additional moiety. In other words, the additional moiety may not be part of a monomer peptide, but may be conjugated thereto.

The linker sequences may be of variable length and/or sequence. It may be understood that the linker sequences must be of sufficient length to allow the helices formed by the monomer peptides to come together into a coiled coil. However, there may be no functional restriction on the maximum length of the linker sequences. Accordingly, the linker sequences may be at least 2 residues in length, such as at least 3, 4, 5, 6, 7, 8, 9, 10, 12, 15, 20, 25 or 30 residues in length.

In other embodiments, for example, the linker sequences may comprise 2-60 residues, more particularly 5-55, 10-50, 15-45, or 20-40, residues. In one embodiment, the linker sequences may comprise 2-50, 3-40, 4-30, 5-20, or 6-15 residues. The nature of the residues present in the linker sequences is not critical. They may be any amino acid, e.g. a neutral amino acid, or an aliphatic amino acid, or alternatively they may be polar or charged or structure-forming e.g. proline. In an embodiment, the linker sequences are flexible linker sequences. Different flexible linkers which might be used are known and widely described in the art. By way of representative example, at least 70% of the amino acids in a linker sequence may be selected from glycine, serine, threonine, alanine, prolinem histidine, asparagine, aspartic acid, glutamine, glutamic acid, lysine, arginine, or a derivative thereof. In some embodiments, the linker is a glycine-rich or glycine-serine-rich sequence.

Each monomer peptide comprises at least one core sequence, as defined above. In some embodiments, one or more of the monomer peptides may comprise 2 or more core sequences, which may be the same or different to each other. As set out above, each core sequence may be flanked on one or both sides by a flanking sequence. Where a core sequence is flanked on both sides, the two flanking sequences may be the same or different. Each monomer peptide may thus comprise 2 or more core sequences, wherein each core sequence is flanked on one or both sides by a flanking sequence. Accordingly, where a monomer peptide comprises 2 or more core sequences, it will be understood that it may further comprise up to 2 flanking sequences for each core sequence. Thus a monomer peptide comprising 2 core sequences may comprise up to 4 flanking sequences. For example, the monomer peptide may be arranged: F-C-F-F-C-F, wherein F represents a flanking sequence and C represents a core sequence.

In some embodiments, a monomer peptide may comprise 2 core sequences and 3 flanking sequences in the arrangement: F-C-F-C-F. In other embodiments, a monomer peptide may comprise 2 core sequences and 2 flanking sequences in the arrangement: F-C-C-F. In other embodiments, a monomer peptide may comprise 2 core sequences and a single flanking sequence, in the arrangement: C-C-F. In some embodiments, a monomer peptide may comprise a single core sequence flanked on both sides by a flanking sequence in the arrangement: F-C-F; or a single core sequence and a single flanking sequence. In some embodiments, the oligomeric protein may comprise only one monomer peptide comprising a flanking sequence. In some embodiments, each of the monomer peptides in the oligomeric protein consists of one core sequence only.

Each core sequence as defined herein has at least 60% identity to SEQ ID NO: 1, which comprises 30 residues, and thus it will be understood that each core sequence may have a length of 18 to 42 residues. In some embodiments, the core sequence in a monomer peptide may have a length of 19-41 residues, such as 20-40, 21-39, 22-38, 23-37, 24-36, 25-35, 26-34, 27-33, 28-32, or 29-31 residues. In some embodiments, the core sequence may comprise 30 residues. There may be more than one core sequence. Each core sequence may be flanked on one or both sides by a flanking sequence. As noted above, said flanking sequence may comprise one or more heptad motifs or parts thereof, one or more additional components, and/or one or more linker sequences. Accordingly, in some embodiments, the monomer peptide as whole may be significantly longer than the core sequence. In some embodiments, the monomer peptide may comprise 24-1000 residues, such as 24-900, 24-800, 24-700, 24-600, 24-600, 24-500, 24-400, 24-300, 24-250, 24-200, 24-150, 24-100, 24-75, 24-50, or 24-40 residues.

The oligomeric protein defined herein has a coiled-coil structure comprising at least 2 monomer peptides. As noted above, the oligomeric protein is based upon the C-terminal stretch of the GCN4 transcription factor. The wild-type C-terminal GCN4 sequence forms a dimeric coiled-coil structure, i.e. a structure comprising 2 monomer peptides. However, it has been observed that by changing the residues at positions a and d within the heptad motif in the individual monomer peptides, the oligomeric state of the protein as whole can be altered, so as to form trimeric or tetrameric structures. In some embodiments, the oligomeric protein is a dimer, a trimer or a tetramer, i.e. the oligomeric protein comprises 2, 3 or 4 monomer peptides. In a preferred embodiment, the oligomeric protein is a trimer.

Each monomer peptide within the oligomeric protein may be the same or different. This includes not only the sequence of the core sequence, but also the presence or absence of (and the sequence of) one or more flanking sequences. In some embodiments, the oligomeric protein may comprise 2 or more monomer peptides having identical core sequences. In some embodiments, the oligomeric protein may comprise 2 or more entirely identical monomer peptides. In some embodiments, all of the monomer peptides in the oligomeric protein may be identical. Whilst it is not required that the monomer peptides within the oligomeric protein are identical to each other, it is preferred that only minimal variations are present between the monomer peptides.

In some embodiments, each monomer peptide within the oligomeric protein may be provided as a separate peptide chain. In this case, each monomer peptide may be seen to be a physically separate subunit of the oligomeric protein complex. Alternatively, in some embodiments, 2 or more of the monomer peptides may be linked together. As outlined above, individual monomer peptides may be linked together by one or more linker sequences into a single peptide chain, i.e. where one end of a first monomer peptide is linked to one end of second monomer peptide. In some embodiments, all of the monomer peptides may be linked into a single peptide chain. In this case, the monomer peptides may be considered as being separate domains of a single-chain, multi-domain protein construct.

Monomer peptides may additionally or alternatively be linked together via chemical-crosslinking, in the form of one or more chemical-cross links between monomer peptides. A number of methods are known in the art for forming covalent bonds between individual peptides to link them together, and any suitable such chemical-crosslinking method may be employed to link together 2 or more monomer peptides within the oligomeric protein. For example, 2 or more monomer peptides may be linked via one or more disulphide bonds between specific cysteine residues in the monomer peptides. Alternatively, they may be stochastically linked by using a cross-linker such as formaldehyde, which is capable of facilitating the formation of covalent bonds with lysine residues present in the monomer peptides. In some embodiments, the oligomeric protein may comprise a combination of monomer peptides that are linked together, in the form of a single peptide chain and/or via chemical-crosslinking, and some monomers that are provided on a separate peptide chain and are unlinked.

The oligomeric protein disclosed herein may be generated synthetically, e.g. by ligation of amino acids or smaller synthetically generated peptides, or by recombinant expression of a nucleic acid molecule encoding said protein or one or more monomer peptides thereof. Such nucleic acid molecules may be generated synthetically by any suitable means known in the art. Thus, the oligomeric protein may be a recombinant or synthesised or artificial oligomeric protein.

The oligomeric protein defined herein is provided as a binding agent for binding to LPS. As noted above, lipopolysaccharides are an integral component in the outer membranes of all Gram-negative bacteria. However, not all Gram-negative bacteria have exactly the same lipopolysaccharides in their outer membranes. As used herein, the term “LPS”, or the term “endotoxin” (which, as noted above, is used interchangeably with “LPS”), refers to any lipopolysaccharide which is present in the outer membrane of a Gram-negative bacteria.

Advantageously, the oligomeric protein defined herein is capable of binding to LPS with extremely high affinity. This high affinity allows the oligomeric protein to bind LPS effectively even when it is present at very low concentrations, such that it can be detected and/or removed. In some embodiments, the oligomeric protein binds to LPS with a K_{D} in the nanomolar or picomolar range, or lower. For example, in some embodiments, the oligomeric protein binds to LPS with a K_{D} of 10 nM or less, such as 5 nM or less, 1000 pM or less, 750 pM or less, 500 pM or less, 250 pM or less, 100 pM or less, 50 pM or less, 10 pM or less, 5 pM or less, 1 pM or less, or 500 fM or less. Accordingly, the oligomeric peptide defined herein may be capable of detecting LPS in a sample where it is present at a concentration of at least 100 pM. In some embodiments, the oligomeric protein is capable of detecting LPS in a sample where it is present at a concentration of at least 75 pM, more particularly at a concentration of at least 50 pM, at least 25 pM, at least 10 pM, at least 5 pM, at least 3 pM, at least 1 pM, at least 750 fM, at least 500 fM, at least 250 fM or at least 100 fM.

Without wishing to be bound by theory, the present inventors believe that the binding of the oligomeric protein defined herein to LPS relies on both the coiled-coil structure of the protein as a whole, and on interactions between LPS and individual residues within the protein. In this regard, it is thought that the presence of positively charged residues within the oligomeric protein may help to increase the affinity of the binding. Again, without wishing to be bound by theory, it is hypothesised that the positively charged residues may be involved in electrostatic interactions with the negatively charged phosphate groups in the lipid A region of LPS. Accordingly, in some embodiments, the oligomeric protein comprises a total of at least 6 cationic residues within the core sequences of the monomer peptides. In some embodiments, the oligomeric protein may comprise a total of at least 7 cationic residues, such as at least 8, at least 9, at least 10, at least 12 or at least 15 cationic residues within the core sequences of the monomer peptides.

The term “cationic residue”, as used herein, includes lysine, arginine, histidine, and any non-genetically coded or modified amino acid residue which carries a positive charge at pH 7.0. Suitable non-genetically coded or modified cationic residues include analogues of lysine, arginine and histidine such as homolysine, ornithine, diaminobutyric acid, diaminopimelic acid, diamionpropionic acid, homoarginine, trimethylysine, trimethylornithine, 4-aminopiperidine carboxylic acid, 4-amino-1-carbamimidoylpiperidine-4-carboxylic acid and 4-guanidinophenylalanine.

The aforementioned cationic residues may be present within the at least one core sequence of a single monomer peptide, or they may be spread across the core sequences of multiple monomer peptides in the oligomeric protein. In some embodiments, each monomer peptide comprises at least 2 cationic residues in the core sequence. In some embodiments, each monomer peptide comprises at least 3, at least 4, or at least 5 cationic residues within the core sequence.

As discussed above, the oligomeric protein interacts with LPS via the lipid A component, and thus also provided herein is the use of an oligomeric protein as defined herein as a binding agent for binding to lipid A. The term “lipid A”, as used herein, refers to the lipid A component of LPS, which comprises two phosphoglucosamine sugar molecules joined by a beta-1,6 linkage, together having four O-linked and two N-linked acyl chains, which are capable of interacting with the outer membrane of Gram-negative bacteria.

In some embodiments, as noted above, the oligomeric protein defined herein may be in the form of a conjugate or a fusion with one or more additional components or moieties. In particular, the oligomeric protein may be conjugated with a detection moiety or an immobilising moiety. The additional moiety may be in the form of a polypeptide and thus the oligomeric protein may be in the form of a fusion protein with a fusion partner. The fusion partner is a separate polypeptide component of the fusion protein to the oligomeric protein. In some embodiments, the oligomeric protein may be immobilised on a solid substrate.

The oligomeric protein may be conjugated with any suitable detection moiety, i.e. any moiety that is capable of providing a signal that can be detected. The detection moiety may be considered to be a label, and may be directly or indirectly detectable. In some embodiments, the oligomeric protein may be conjugated with a detection moiety that is directly detectable. A moiety that is directly detectable is one that can be directly detected without the use of additional reagents. For example, suitable detection moieties which are directly detectable may include fluorescent molecules (e.g. fluorescent proteins or organic fluorophores), colorimetric moieties (e.g. coloured molecules or nanoparticles), particles, for example gold or silver particles, quantum dots, radioisotopic labels, chemiluminescent molecules, and the like. In particular, any spectrophotometrically or spectroscopically detectable label may be used in a directly detectable moiety. The detectable label may be distinguishable by colour, but any other parameter, e.g. size, charge, etc. may be used.

An indirectly detectable moiety is one that is detectable by employing one or more additional reagents, e.g., where the moiety is a member of a signal producing system made up of two or more components. For example, the detection moiety may comprise an enzyme such as horseradish peroxidase (HRP) capable of catalysing a reaction which produces a detectable signal, such as a colour change. Accordingly, upon contacting the detection moiety with the substrate for the enzyme, the reaction would proceed and the detectable signal would be generated.

The oligomeric protein may be in the form of a fusion protein with a fusion partner. In some embodiments, the fusion partner may be a detectable moiety, i.e. the oligomeric protein in the form of a conjugate with a detectable moiety may be considered to be equivalent to the oligomeric protein in the form a fusion protein with a detectable fusion partner. However, the oligomeric protein may be in the form of a fusion protein with a fusion partner other than a detectable moiety. In principle, the fusion partner may be any polypeptide, provided that the oligomeric protein is still capable of functioning as binding agent for binding to LPS.

In some embodiments, the oligomeric protein may be immobilised on a solid substrate (i.e. a solid phase or solid support). This immobilisation may be achieved in any convenient way. Thus the manner or means of immobilisation and the solid substrate may be selected, according to choice, from any number of immobilisation means and solid substrates as are widely known in the art and described in the literature. In some embodiments, the oligomeric protein may be conjugated with an immobilising moiety to facilitate the immobilisation. The immobilising moiety may be directly bound to the solid substrate, (e.g. chemically cross-linked). In some embodiments, for example, the immobilising moiety may comprise a cysteine residue which is capable of being coupled to a cysteine residue on the substrate in the form of a disulphide bridge. In some embodiments, the immobilising moiety may be bound to the substrate more indirectly, by means of a linker group or by one or more intermediary binding groups. In some embodiments, the immobilising moiety may be, for example, an affinity binding partner, e.g. biotin or a hapten, capable of binding to its binding partner, i.e. a cognate binding partner, e.g. streptavidin or an antibody, which is provided on the solid substrate. Thus, the oligomeric protein, via the immobilising moiety, may be covalently or non-covalently linked to the solid substrate. The linkage may be a reversible (e.g. cleavable) or irreversible linkage. In some embodiments, the linkage may be cleaved enzymatically, chemically, or with light, e.g. the linkage may be a light-sensitive linkage.

In some embodiments, the interaction between the oligomeric protein and the solid substrate must be robust enough to allow for washing steps, i.e. the interaction between the oligomeric protein and the solid substrate is not disrupted (significantly disrupted) by the washing steps. For instance, in one embodiment, less than 5% of the oligomeric protein is removed or eluted from the solid substrate with each washing step. In one embodiment, less than 4, 3, 2, 1, 0.5 or 0.1% of the oligomeric protein is removed or eluted from the solid substrate with each washing step.

The solid substrate may be any of the well-known substrates or matrices which are currently widely used or proposed for immobilisation, separation etc. These may take the form of particles (e.g. beads which may be magnetic, para-magnetic or non-magnetic), sheets, gels, filters, membranes, fibres, capillaries, slides, arrays, chips or microtitre strips, tubes, plates or wells etc.

In some embodiments, the oligomeric protein is immobilised on a bead or resin, or in or on a well or vessel, or a column or filter material, or on a surface of a detection device.

The substrate may be made of glass, silica, latex, apatite, or a polymeric material. In some circumstances, materials having a high surface area may be particularly suitable. Such substrates may have an irregular surface and may be for example porous or particulate, e.g. particles, fibres, webs, sinters or sieves. Particulate materials, e.g. beads are useful due to their greater binding capacity, particularly polymeric beads. It will be understood that these beads may be provided in any suitable arrangement, as known in the art. For example, the beads may be packed into a column, such as a filtration column.

Conveniently, a particulate solid substrate used according to the present disclosure may comprise spherical beads. The size of the beads is not critical, but they may for example be of the order of diameter of at least 1 μm. In one embodiment, the beads may have a diameter of at least 2 μm. In one embodiment, the beads may have a maximum diameter of not more than 10, and e.g. not more than 6 μm. Monodisperse particles, that is those which are substantially uniform in size (e.g. size having a diameter standard deviation of less than 5%) have the advantage that they provide very uniform reproducibility of reaction. Representative monodisperse polymer particles may be produced by the technique described in U.S. Pat. No. 4,336,173.

In some embodiments, the solid substrate may be a resin, such as an amylose resin. The resin may be provided in any suitable form, such as a spin column filter or a flow column. In some embodiments, the oligomeric protein may be immobilised in or on a well or vessel, such as a multiwell plate.

In some embodiments, the oligomeric protein may be immobilised on a surface of a detection device, such as a chip or a microarray. In this regard, the oligomeric protein may form a capture array or a biosensor capable of binding to and detecting LPS. In some embodiments, the oligomeric protein may be immobilised on a surface plasmon resonance (SPR) chip. Biosensors which are capable of measuring a signal corresponding to the binding of a target to an immobilised capture protein are well known in the art, and the oligomeric protein defined herein may be provided in any such suitable arrangement.

Thus, it can be seen that the use of the oligomeric protein defined herein as a binding agent for binding to LPS may comprise the use of the oligomeric protein to detect and/or to remove LPS in or from a sample.

Accordingly, uses of the oligomeric protein as defined and described herein include particularly in vitro uses, that is the LPS is bound, detected or removed in vitro.

In this regard, provided herein is a method of binding LPS, the method comprising contacting the LPS, or a sample containing LPS, with an oligomeric protein as defined herein, to allow the protein to bind to the LPS to form a protein-lipopolysaccharide complex. It will be understood that the disclosures above in relation to the oligomeric protein for use in binding LPS apply equally to the methods for binding LPS involving the same oligomeric protein.

In an embodiment, the method is an in vitro method.

The term “sample” as used herein includes any sample that may contain, or may be contaminated with LPS, or that it may be desired to test. This includes clinical samples derived from patients or subjects more generally, environmental samples, and samples of products which are to be tested for endotoxin contamination. A clinical sample derived from a patient may be any sample of body fluid or tissue, e.g. a blood sample, a lymph sample, a saliva sample, a urine sample, a faeces sample, a cerebrospinal fluid sample or any other appropriate biological sample taken from a patient. In a preferred embodiment, the clinical sample is a blood sample.

A sample of a product which is to be tested for endotoxin contamination may be a sample derived from any product which is suspected of being contaminated with endotoxins, and particularly any such product which is intended for human consumption or for interaction with humans. This includes, for example, products from pharmaceutical and medical industries, such as reagents, medical devices, equipment, consumables, medicines, vaccines, etc. Similarly, samples may also be derived from products in food and beverage industries, or from environmental samples, such as drinking water, ground water, etc.

In one embodiment, the sample may be a liquid sample comprising a portion of the product to be tested, though it may also be a sample derived from the surface of a product, where it is desired to test a solid product, such as a medical device, or a surface, such as a surface in an operating theatre or another sterile environment, for endotoxin contamination. This may include for example swabs or washes taken from the surface of a product.

In some embodiments, the method of binding LPS is a method of detecting the presence of LPS in a sample, wherein the method comprises:
(a) contacting the sample with an oligomeric protein as defined herein, to allow the protein to bind to the LPS to form a protein-lipopolysaccharide complex; and
(b) detecting the presence of a protein-lipopolysaccharide complex.

In some embodiments, the method of binding LPS may be a method of detecting the presence of Gram-negative bacteria in a sample which is suspected to contain Gram-negative bacteria, wherein the method comprises:
(a) contacting the sample with an oligomeric protein as defined herein, to allow the protein to bind to the LPS in the outer membrane of the Gram-negative bacteria to form a protein-lipopolysaccharide complex; and
(b) detecting the presence of a protein-lipopolysaccharide complex.

It will be understood that the step of detecting the protein-lipopolysaccharide complex may be done by any suitable means known in the art. The protein-lipopolysaccharide complex may be directly or indirectly detected. An appropriate method to detect the protein-lipopolysaccharide complex may be chosen, depending on the method by which the sample is contacted with the oligomeric protein.

The step of contacting the sample with the oligomeric protein may involve applying the sample to a substrate to which the oligomeric protein has been immobilised, as outlined above, wherein the substrate is arranged such that the binding of the sample to the oligomeric protein can be measured. In some embodiments, for example, the oligomeric protein may be immobilised on a surface of a detection device as outlined above, such as an SPR chip or another suitable biosensor, which is capable of detecting interactions between the sample and the oligomeric protein. Accordingly, the step of contacting the sample with the oligomeric protein may involve applying the sample to a solid substrate on which the oligomeric protein has been immobilised.

In other embodiments, for example, the oligomeric protein may be immobilised in a well of a multiwell plate in order to form an LPS assay. Such assays are well known in the art; when the sample is applied to a plate comprising the immobilised oligomeric protein, any LPS which is present in the sample will be bound by the oligomeric protein, and the other components in the sample can be washed away. Accordingly, the step of contacting the sample with the oligomeric protein may involve applying the sample to a multiwell plate on which the oligomeric protein has been immobilised. Moreover, the method of detecting the presence of LPS in a sample may further comprise a step of washing the protein-lipopolysaccharide complex before the step of detection so as to remove unbound components of the sample, and therefore improve the accuracy of the method. Suitable reagents and protocols for such washing steps are well known in the art. The protein-lipopolysaccharide complex which is retained in the plate can then be detected using any suitable detection moiety which is capable of binding to LPS. The detection moiety may be directly or indirectly detectable. As is outlined in more detail below, the present inventors adapted an ELISA-like assay using tailspike proteins (ELITA) of Salmonella phages originally reported by Schmidt et al, 2016, to detect LPS. This assay uses a tailspike protein which is capable of binding to LPS and which comprises an N-terminal StrepTag, and a streptavidin-conjugated horseradish peroxidase, to detect the protein-lipopolysaccharide complex. When the enzyme substrate 2,2′-azino-bis 3-ethylbenzothiazoline-6-sulphonic acid (ABTS) is added to the plate, a detectable colour change is induced. Accordingly, it can be seen that the step of detecting the presence of a protein-lipopolysaccharide complex may comprise contacting the protein-lipopolysaccharide complex with a detection moiety which is capable of binding to LPS and which comprises an enzyme capable of catalysing a reaction which produces a detectable signal, and with an appropriate substrate to induce such a detectable signal.

In some embodiments, the oligomeric protein may be in the form of a conjugate comprising a detection moiety itself, as outlined above. Accordingly, the protein-lipopolysaccharide complex may be detected by detecting a signal from the detection moiety which is conjugated with the oligomeric protein. This may be done by any method which is appropriate for detecting a signal from the detection moiety in question, for example using fluorescence microscopy to observe a fluorescent label which is conjugated to the oligomeric protein.

In some embodiments, the method of binding LPS is a method of removing LPS from a sample, wherein the method comprises:
(a) contacting the sample with an oligomeric protein as defined herein, to allow the protein to bind to the LPS to form a protein-lipopolysaccharide complex; and
(b) separating the protein-lipopolysaccharide complex from the sample.

Again, it will be understood that the step of separating the protein-lipopolysaccharide complex from the sample may be done by any suitable means known in the art, and that this will depend on the way in which the sample is contacted with the oligomeric protein.

In some embodiments, the oligomeric protein may be immobilised on a solid substrate, and thus the step of contacting the sample with the oligomeric protein may comprise applying the sample to the solid substrate on which the oligomeric protein is immobilised. As noted above, the oligomeric protein may be immobilised on any suitable substrate known in the art. In particular, the solid substrate may be in the form of particles (e.g. beads), filters or columns. Again, suitable reagents and protocols for using such substrates to separate a bound target molecule from a sample are well known in the art.

As noted above, the oligomeric protein may be immobilised on to beads, which may be magnetic. The term “magnetic” as used herein means that the substrate is capable of having a magnetic moment imparted to it when placed in a magnetic field, and thus is displaceable under the action of that field. In other words, a substrate comprising magnetic particles may readily be removed by magnetic aggregation, which provides a quick, simple and efficient way of separating the protein-lipopolysaccharide complex from the sample, once the complex has been formed.

In another embodiment, for example, the oligomeric protein may be immobilised on a resin which is packed into a column. In this example, when the sample is contacted with the oligomeric protein, i.e. when the sample is applied to the column, the LPS will be bound by the oligomeric protein and retained in the column, and the rest of the sample will flow through the column. In some embodiments, the method may comprise multiple steps of contacting the sample with the oligomeric protein, in order to ensure that all of the LPS is bound, i.e. the sample may be applied to the column several times. In addition, the method may comprise a step of washing the protein-lipopolysaccharide complex, before the step of separation, to avoid inadvertently removing other components from the sample in addition to LPS, i.e. the column may be washed with an appropriate reagent.

It is advantageous when binding LPS, detecting the presence of LPS in a sample, or removing LPS from a sample, if the reagents involved in the binding, detecting or removing, particularly the oligomeric protein, can be reused. Accordingly, the methods disclosed herein may further comprise a step of contacting the protein-lipopolysaccharide complex with at least one non-denaturing detergent, in order to remove LPS from the oligomeric protein, i.e. to disrupt the protein-lipopolysaccharide complex, so that the oligomeric protein can be reused.

In this regard, provided herein is a kit for use as a binding agent for LPS as defined herein, or for use in the methods defined herein, said kit comprising:
(i) an oligomeric protein as defined herein; and
(ii) at least one non-denaturing detergent.

Non-denaturing detergents are well known in the art, and the skilled person may use any suitable non-denaturing detergent. For example, the at least one non-denaturing detergent may be selected from non-ionic, anionic, cationic or zwitterionic detergents, or any combination thereof. In this regard, the at least one non-denaturing detergent may have a headgroup selected from a linear polyethylene glycol (PEG) group, a polysorbate group, a beta-glycosidic sugar group, an N-methylglucamine group, an N-oxide group, a dimethylammonium-1-propanesulfonate group, a carboxylic acid group, a sulfate group, or a quaternary amine group. The at least one non-denaturing detergent may be selected from CHAPS, zwittergent 3-12, polysorbate 80, polysorbate 20, triton X-100, or any combination thereof. In some embodiments, the at least one non-denaturing detergent may be a mixture of non-denaturing detergents. In some embodiments, the mixture of non-denaturing detergents comprises or consists of CHAPS, zwittergent 3-12, polysorbate 80, polysorbate 20 and triton X-100.

It will be understood that the detergent should be present at a sufficient concentration to disrupt the protein-lipopolysaccharide complex, without being at such a high concentration that function of the oligomeric protein is permanently impaired. In some embodiments, the detergent may be present at a total concentration, i.e. the concentration of all detergents present, of at least 0.1% (w/w) or 0.1% (v/v). In some embodiments, the concentration of detergent may be at least 0.15% (w/w) or at least 0.15% (v/v), such as at least 0.2% (w/w) or at least 0.2% (v/v), at least 0.25% (w/w) or at least 0.25% (v/v), or at least 0.5% (w/w) or at least 0.5% (v/v). In one embodiment, the at least one non-denaturing detergent comprises a combination of 0.05% (w/w) CHAPS, 0.05% (w/w) zwittergent 3-12, 0.05% (v/v) tween 80, 0.05% (v/v) tween 20, and 0.05% (v/v) triton X-100.

In a further embodiment, provided herein is a product comprising an oligomeric protein immobilised on a solid substrate, wherein the oligomeric protein is as defined herein. The solid substrate may be any solid substrate disclosed herein. That is to say that the disclosures above in relation to the use of the oligomeric protein, wherein the oligomeric protein is immobilised on a solid substrate, apply equally in the context of the product comprising the oligomeric protein immobilised on a solid substrate. In this regard, the solid substrate may be a sheet, gel, filter, membrane, fibre, capillary, slide, array, chip, microtitre strip, tube, plate or well. In particular, the oligomeric protein may be immobilised on the surface of a detection device, such as an SPR chip or a biosensor.

It will be seen from the disclosure above that the oligomeric protein described herein provides an alternative binding agent for binding LPS, which may address a number of the issues with known methods for binding and detecting LPS. In particular, the oligomeric protein described herein is capable of dissolving LPS aggregates. Accordingly, the oligomeric protein can reduce the impact of LPS masking caused by aggregation, and therefore effectively increase the measurable concentration of LPS in a sample. This oligomeric protein thus supports a method of detection of LPS which is capable of detecting low concentrations of LPS.

In addition, this detection method avoids the problems which are associated with the LAL assay, such as the expensive and unsustainable harvesting of the amebocyte lysate. Moreover, this method also avoids any potential problems connected to the use of Factor C, which may also exist with recombinant variants of the LAL assay.

### FIGURES

The invention will now be described in more detail in the following non-limiting Examples with reference to the following drawings:

FIG. 1 shows the structure of the GCN4-pII trimer adapted from PDB-ID 2YO0 (Hartmann et al., 2012). (A) Side view. (B) Front view with core isoleucine residues in positions a and d colored green.

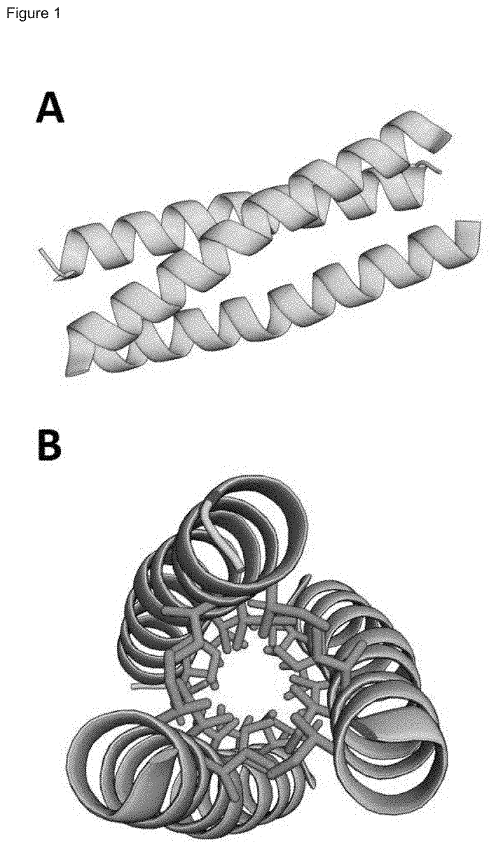

FIG. 2 shows a schematic version of the general structure of LPS, based on LPS from S. typhimurium. The Lipid A moiety (insert) consists of two phosphoglucosamines with four O-linked and two N-linked acyl chains embedded in the outer membrane. The core oligo saccharide (COS) is linked to Lipid A via a glycosidic bond, and the O-antigen linked to the penultimate COS sugar. The O-antigen consists of a four-sugar repeat varying between 4 and 40 repeat units, with an average of 30 repeats (Peterson and McGroarty, 1985). Lipid A and the two proximal 3-Deoxy-D-manno-oct-2-ulosonic acid (KDO) sugars are highly conserved among Gram-negative species, while the rest of COS and O-antigen are conserved among bacterial species and serotypes, respectively.

FIG. 3 shows SPR binding curves following injection of different LPS components to immobilized K9-GCN4-PII. Vertical lines indicate start and end of injection. (A) shows the injection of whole LPS. (B) shows the injection of rough LPS lacking O-antigen. (C) shows the injection of deep rough LPS, lacking all sugars except the two proximal KDOs. (D) shows the injection of LPS polysaccharide lacking Lipid A.

FIG. 4 shows a graph of SPR difference values at end of injection normalized for the molar concentrations of the ligands, for each of the LPS components tested in FIG. 3.

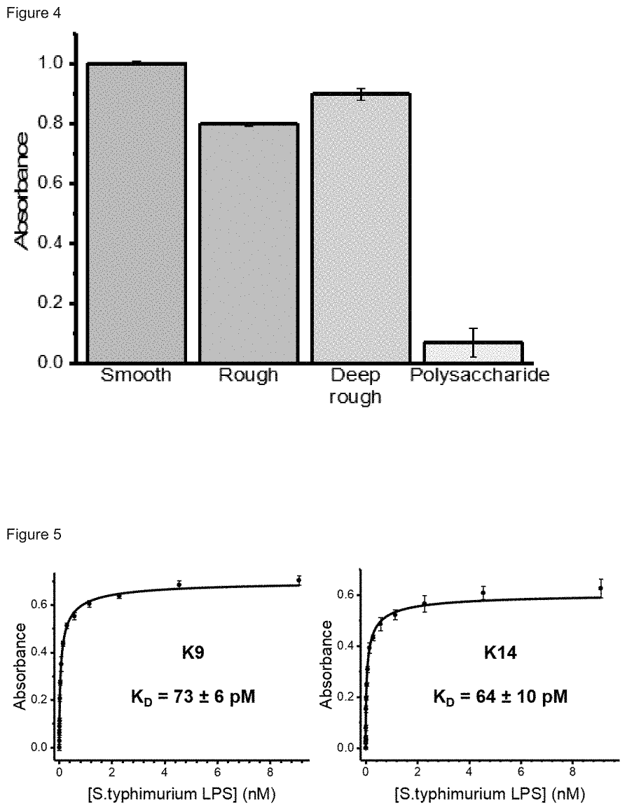

FIG. 5 shows ELITA binding curves of LPS to the two GCN4-containing constructs K9-His (left) and K14-His (right).

FIG. 6 shows TEM images of LPS alone (top) and LPS with GCN4-pII (bottom) at 4k (left) and 8k (right) magnification.

FIG. 7 shows a schematic overview of the constructs that were produced. Constructs derived from SadA were originally described in Alvarez et al. (Alvarez et al., 2008) and Hartman et al. (Hartmann et al., 2012). The andreinlvpas construct was originally described by Deiss et al. (Deiss et al., 2014). The GCN4 construct was synthesized by GenScript (GenScript Biotech Corp).

FIG. 8 shows SPR Fc1, Fc2, and Fc1-F2 curves for immobilized K9 with different S. typhimurium LPS components. (A) is with smooth LPS. (B) is with rough LPS. (C) is with deep rough LPS. (D) is with polysaccharide derived from LPS.

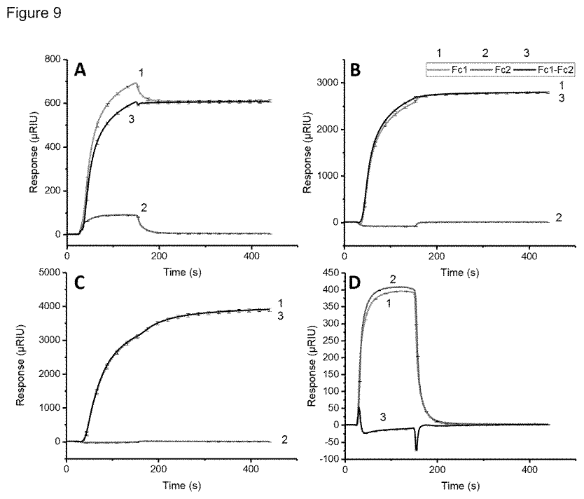

FIG. 9 shows SPR Fc1, Fc2, and Fc1-F2 curves for immobilized K14 with different S. typhimurium LPS components. (A) is with smooth LPS. (B) is with rough LPS. (C) is with deep rough LPS. (D) is with polysaccharide derived from LPS.

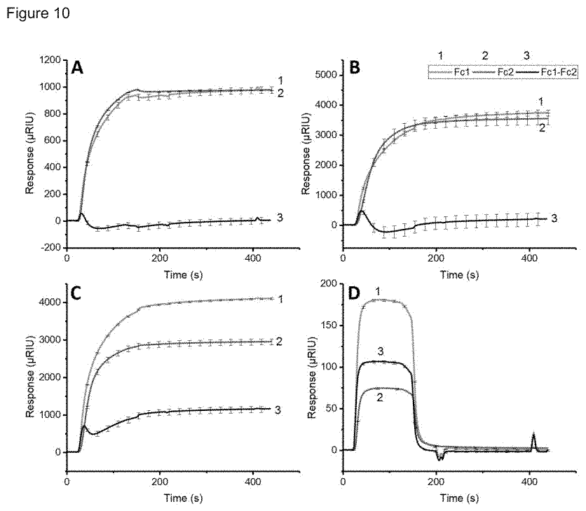

FIG. 10 shows SPR Fc1, Fc2, and Fc1-F2 comparison curves for immobilized K3 (Fc1 channel) and K3-His (Fc2 channel) with different S. typhimurium LPS components. (A) is with smooth LPS. (B) is with rough LPS. (C) is with deep rough LPS. (D) is with polysaccharide derived from LPS.

FIG. 11 shows the absolute values (top) and the set-up and negative controls (bottom) of the ELITA experiments with K9 and K14. SadA=Salmonella component K9 or K14. BSA=Bovine serum albumin. TSP=phage tailspike protein. ST-HRP=streptactin conjugated horseradish peroxidase.

FIG. 12 shows the CD spectra of GCN4-pII alone and in the presence of LPS. It can be seen that there is minimal variation in the secondary structure composition of GCN4-pII before and after binding to LPS.

FIG. 13 shows a graph of the results of an LAL assay demonstrating the masking effect of GCN4-pII at concentrations ranging from 10 to 0.1 μM spiked with 0.5 EU/mL LPS. Optimal masking was observed at 1 μM GCN4-pII where the masking effect was 89% of total signal.

FIG. 14 shows the fingerprint region of a 2D ¹H-¹H TOCSY NMR spectrum of GCN4-pII. All 29 expected spin systems were well resolved and assignable without indications of peak splitting, indicating that the sample is homogenous in solution.

FIG. 15 shows a graph of the results of a GCN4-pII based ELISA using biotinylated LPS for detection.

FIG. 16 shows a graph of the results of an LAL assay using the same samples as in the assay of FIG. 15.

FIG. 17 shows a graph comparing the results of the GCN4-pII based ELISA and the LAL assay.

FIG. 18 shows SPR binding curves for various LPS types and for PBS-E, the running buffer, as a control.

FIG. 19 shows the phylogenetic distribution of the LPS variants used in Example 5. This figure is adapted from Bern and Goldberg, 2005.

### EXAMPLES

Methods

Expression and Purification of Proteins

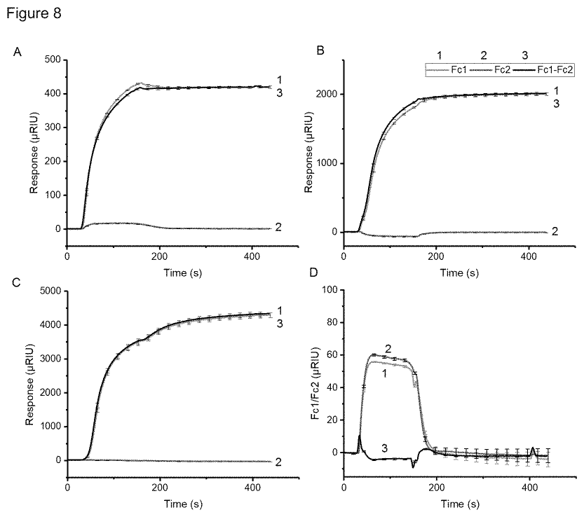

Salmonella adhesin A (SadA) constructs (as shown in FIG. 7) flanked by GCN4 adaptors were produced as described earlier (Alvarez et al., 2008; Hartmann et al., 2012). Tranformed BL21-Gold(DE3) were grown in 2 L ZYP-5052 autoinduction medium (Studier, 2005), and overexpression induced by adding 200 ng/mL anhydrotetracycline (AHTC) at OD600=0.6 followed by expression overnight at 30° C. The cells were pelleted at 6000×g (Beckman JLA 8.1000 rotor) for 30 minutes and resuspended in 20 mL Tris/HCl pH 7.4, 40 mM NaCl, 5 mM MgCl2 containing 200 μL EDTA-free Protease Inhibitor Cocktail (Merck) and DNase I. Following resuspension, the cells were lysed by French press and the resulting lysate diluted in 50 mL equilibration buffer (20 mM Tris/HCl pH 7.9, 5 M guanidine hydrochloride, 0.5 M NaCl, 10% glycerol) and incubated at room temperature for 1 hour while stirring, followed by centrifugation at 75 000×g (Beckman Ti 70 rotor) for 1 hour to remove any undissolved particulates. The resulting solution was loaded onto a 20 mL Ni Sepharose excel columns (GE Life Sciences) pre-equilibrated with equilibration buffer. Following application of the sample, the column was washed with 4 column volumes equilibration buffer and eluted using a 0-100% gradient elution buffer (20 mM Tris/HCl, pH 7.5, 5 M guanidine hydrochloride, 0.5 M NaCl, 10% glycerol, 500 mM imidazole). The eluted fractions were analyzed by SDS-PAGE, and fractions containing the protein of interest was pooled and refolded by dialyzing twice against 2 L refolding buffer (20 mM MOPS pH 7.4, 350 mM NaCl, 10% glycerol) over night.

LPS Production and Purification

LPS was produced by inoculating a 20 mL lysogeny broth (LB) preculture from a single bacterial colony (see Table 3 below for strains used) and grown over night at 37° C.

TABLE 3

| Strain | LPS name | Type | Notes |
|---|---|---|---|
| E. coli BL21 | BL21 LPS | Rough | Common lab strain |
| | | | lacking O-antigen |
| S. anatum | S. anatum LPS | Smooth | Exact structure unknown |
| S. Typhimurium | Smooth LPS | Smooth | |
| S. Typhimurium | WaaC LPS | Rough | Deletion of heptosyl- |
| ΔwaaC (ΔLPS) | | | transferase WaaC, |
| | | | responsible for transfer |
| | | | of heptose to the |
| | | | Kdo-moiety |
| | | | of LPS-precursor |
| S. Typhimurium | WaaJ LPS | Rough | Deletion of |
| ΔwaaJ | | | glycosyltransferase |
| | | | WaaJ, responsible |
| | | | for transfer of the |
| | | | penultimate glucose to |
| | | | the core-oligo LPS |
| S. Typhimurium | Waal LPS | Rough | Deletion of WaaL ligase, |
| ΔwaaL | | | responsible for ligating |
| | | | O-antigen to |
| | | | core-oligo saccharide |

6×1 L cultures in 2 L baffled flasks were inoculated from the preculture and grown over night at 37° C. on a shaker. The bacteria were harvested by centrifuging at 6000×g (Beckman JLA 8.1000 rotor) for 30 minutes. Further purification followed two different methods depending on the type of LPS.

Rough LPS was purified following the protocol described by Galanos et al. (Galanos, Luderitz and Westphal, 1969), using phenol-chloroform-petroleum ether extraction. Following harvest, the bacterial pellet was washed 3 times with 40 mL ethanol and once with acetone, then left over night under an airflow. The dried out pellet was homogenized using a mortar and pestle and dissolved in a 40 ml mixture of 90% (W/V) liquid phenol, chloroform, and petroleum ether in a ratio of 2:5:8. After one hour incubation on a shaker, the undissolved material was pelleted at 4200×g for 15 minutes and the supernatant collected. Chloroform and petroleum ether was removed under an airflow for 4 hours or until the phenol started crystallizing. The solution was resuspended by heating to 40° C., and water added dropwise (3×5 drops) under stirring until the LPS precipitated. The LPS was pelleted at 4200×g for 15 minutes, and more water added to the supernatant to collect any residual LPS. The pellets were washed two times with 10 mL 80% (W/V) phenol, and taken up in 20 mL milliQ-water before centrifugation at 100 000×g (Beckman, MLA-50 rotor) for one hour. The final pellet was taken up in 50 mL MilliQ-water and lyophilized to yield pure LPS.

Smooth LPS was purified following the protocol described by Darveau et al. (Darveau and Hancock, 1983). The bacteria was washed twice and resuspended in 40 mL 10 mM Tris-HCl pH 8.0, 2 mM MgCl2, and lysed by french press followed by additional disruption by sonication. The resulting suspension was incubated with 200 μg/mL DNase I, 50 μg/mL RNase A overnight while stirring at 37° C. To 15 mL suspension, 5 mL 0.5 M EDTA in 10 mM Tris-HCL pH 8.0, 2.5 mL 20% SDS in 10 mM Tris-HCl pH 8.0, and 2.5 mL 10 mM Tris-HCl pH 8.0 were added, and the LPS micelles further disrupted by sonication. The solution was centrifuged at 39 000×g (Sorval, SS-34 rotor) for 30 minutes at 20° C. to pellet undissolved cell components, the supernatant frozen and lyophilized. The lyophilized crude extract was dissolved in a modicum of water, and the LPS precipitated with 2 volumes of ice cold ethanol and 0.375 M MgCl2 at −40° C. overnight. The precipitated LPS was centrifuged at 11 000×g (Sorvall, SLA-3000 rotor) for 15 minutes at 4° C., and the resulting pellet resuspended in the same volume 90% (W/V) phenol at 65° C. for 30 min while stirring. The mixture was centrifuged at 4000×g for 10 min to accelerate phase separation. The water phase was collected, and the phenol phase extracted once more with water. The pooled water phases were pooled, and phenol extracted using ¼ the volume of chloroform. The water phase was placed under an airflow overnight to evaporate any residual organic solvent, and dialysed against MQ-water for 3 days using a 500 MWCO dialysis membrane. The dialyzed LPS was frozen and lyophilized to yield pure LPS.

The purity of the LPS products which were isolated was controlled by tricine-SDS-polyacrylamide gel electrophoresis (Marolda et al., 2006).

Preparation of O-Antigen Polysaccharides

Polysaccharides were isolated from wild type S. typhimurium (smooth) LPS by mild acid hydrolyzation of the glycosidic bond connecting LipidA to the proximal KDO sugar (Raetz and Whitfield, 2002a). 4-5 mg/mL S. typhimurium LPS was dissolved in 10% acetic acid and incubated at 100° C. for 1 hour. The resulting Lipid A was removed from the solution by centrifugation at 10 000×g for 30 minutes at 4° C., the supernatant containing the polysaccharide was frozen and lyophilized overnight.

ELISA-Like Tailspike Adsorption (ELITA) Assay

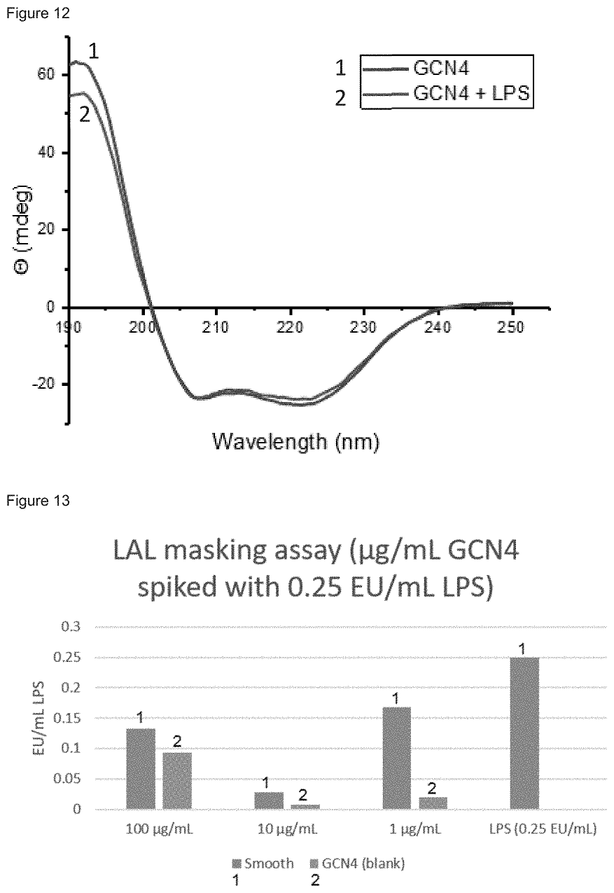

The ELITA assay was first described by Schmidt et al (Schmidt et al., 2016) using whole bacteria. Here, we modified the assay for use with purified proteins in a Nunc MaxiSorp 96-well flat-well plate (as shown in FIG. 11). The wells were saturated by incubating with 100 μl 10 μg/mL of either K9-His or K14-His in PBS-buffer overnight. Following a 2 hour blocking step with 2% bovine serum albumin (BSA) in PBS, 100 μL dilutions of Salmonella typhimurium LPS ranging from 200 μg/mL to 0.0023 μg/mL were added as a binding partner and incubated for 1 hour. 100 μL P22 tailspike protein (P22TSP) with an N-terminal Strep-tag®II (IBA) was added for one hour, before the wells were finally incubated with 100 μL 1:10 000 StrepTactin-conjugated horse radish peroxidase (IBA, Gottingen) for one hour, and developed with 2,2′-azino-bis 3-ethylbenzothiazoline-6-sulphonic acid (ABTS, Sigma-Aldrich) for 30-60 min and read at 407 nm using a plate reader. The wells were washed 3 times with 150 μL PBS-buffer containing 0.1% BSA between each of the above steps (Tween-20 was omitted for these experiments since it interfered with the assay). The average background signal (0 μg/mL LPS) was subtracted from each average signal, propagation of error was calculated by adding the individual standard deviations for the triplicates to the baseline in quadrature (δQ=\sqrt{(δa)²+(δb)²+ . . . +(δz)²}) where δQ is the uncertainty of a combination of sums Q). The dose-response curve and dissociation constant K_{D}, was calculated by curve fitting the data to the Hill equation as follows:

$$Y = \frac{{{Y}_{max} [ L ]}^{n}}{{( {K}_{D} )}^{n} + {[ L ]}^{n}}$$

where Y denotes the fraction of occupied receptor binding sites, Yₘₐₓ the maximal binding, [L] the concentration of free ligand, and n the number of binding sites. Although each construct carried two GCN4-PII motifs, n was treated as being equal to 1, since they are localized at opposite ends of the protein, and thus are not expected to cooperate. The average molecular weight of smooth S. typhimurium LPS was calculated to 22 kDa assuming an average of 30 O-antigen repeats polysaccharide structure as reported (Peterson and McGroarty, 1985; Raetz and Whitfield, 2002b; Schmidt et al., 2016).

Surface Plasmon Resonance Experiments (Examples 1 to 3)

All SPR-experiments were conducted on a Reichert 2SPR system at ambient temperature using PBS-E (PBS pH 7.4+5 mM EDTA) running buffer. The proteins were diluted to 50 μg/mL in 20 mM sodium acetate buffer pH 4.5 and immobilized to a CMD200 sensor chip (Xantec Bioanalytics, Duesseldorf, Germany) using NHS-EDC amine coupling (Fischer, 2010) to a response of 2000-9 000 μRIU. Following a comparison of different reference compounds (ethanolamine, BSA, casein, and skimmed milk) (Péterfi et al., 2000), ethanolamine was chosen as the standard coating for the reference channel for all experiments.

All ligands were solubilized to 1 mg/mL in running buffer by extrusion (21 passes through a 100 μm filter at 70° C.). The experiments were performed at 50 μL/min flowrate in triplicates. Each sample was injected over both measurement and reference channel for 90 s followed by 300 s dissociation. The chip was regenerated by 2×30 s injection of regeneration buffer (0.05% (w/w) CHAPS, 0.05% (w/w) zwittergent 3-12, 0.05% (v/v) tween 80, 0.05% (v/v) tween 20, and 0.05% (v/v) triton X-100) (Andersson, Areskoug and Hardenborg, 1999). The measurement data was exported to TraceDrawer (RidgeView instruments lab) for processing, and final curves generated using Origin (OriginLab corporation). The signal for each construct was normalized to K9 using the following formula S=

$${S}_{0} ( \frac{\frac{R}{MW}}{\frac{{R}_{K 9}}{{MW}_{K 9}}} )$$

where S is the normalized signal, S0

Surface Plasmon Resonance Experiments (Examples 4 and 5)

SPR experiments were conducted on a Nicoya OpenSPR system at ambient temperature using PBS-E (PBS pH 7.4+5 mM EDTA) running buffer. SadA K9 was diluted to 50 μg/mL in 10 mM sodium acetate buffer pH 4.5 and immobilized to Carboxyl Sensor (OpenSPR) using NHS-EDC amine coupling (Fischer, 2010) to a response of 700 RU.

All ligands were solubilized to 1 mg/mL in running buffer by extrusion (21 passes through a 100 μm filter at 70° C.). The experiments were performed at 35 μL/min flowrate in triplicates. Each sample was injected over both measurement and reference channel for 125 s followed by 300 s dissociation. The chip was regenerated by 125 s injection of regeneration buffer (0.05% (w/w) CHAPS, 0.05% (w/w) Zwittergent 3-12, 0.05% (v/v) Tween 80, 0.05% (v/v) Tween 20, and 0.05% (v/v) Triton X-100) (Andersson et al., 1999). The measurement data was exported to TraceDrawer (RidgeView instruments lab) for processing, and final graphs were generated using Origin (OriginLab corporation).

Electron Microscopy

Samples were adhered to a measuring grid, stained for one minute with 1% uranyl acetate and embedded in 1.8% methylcellulose/0.4% uranyl-acetate. Images were recorded in a Philips CM100 transmission electron microscope at 80 kV using a Olympus Quemesa camera.

Limulus Amebocyte Lysate (LAL) Assay

The masking effect of GCN4-PII on LPS was tested using the LAL-assay (Pierce, Thermofisher). GCN4-PII concentrations ranging from 200 μg/mL-20 pg/mL was spiked with 0.5 endotoxin units per mL LPS (EU/mL), and developed following the provided protocol.

Circular Dichroism

Spectra were recorded using a Jasco J-810 spectropolarimeter (Jasco International Co). Measurements were done using a 1.0 cm path length quartz cuvette. Each samples was scanned five times in the range of 190 to 250 nm with a scanning rate of 50 nm/min with a bandwidth of 0.5 nm. Spectra were recorded with a GCN4-pII to LPS ratios of 0, 0.5, 1, 3, and 9 in 10 mM Tris pH 7.4 at 37° C. The approximate α-helical content of the peptide was calculated using K2D2.

Nuclear Magnetic Resonance (NMR) Spectroscopy

NMR experiments for assignment were carried out in Bel-Art™ SP Scienceware™ 5 mm O.D. Thin Walled Precision NMR Tubes containing 450 μL 1.5 mM synthetic FMet-GCN4-PII (Genscript, China) in 50 mM NaCl, 7% D2O, and 0.2 mM 4,4-dimethyl-4-silapentane-sulfonic acid (DSS). Spectra were acquired at 308 K on a Bruker Avance II 600 MHz NMR spectrometer equipped with a 5 mm 1H/13C/15N-cryoprobe. DSS was used as internal chemical shift standard, and 13C and 15N was referenced using frequency ratios as described (Wishart et al., 1995). The following spectra were collected for assignment: 13C-1H-HSQC, 15N-1H-HSQC, 1H-1H COSY, 1H-1H TOCSY using a mixing time of 60 and 80 ms, and 1H-1H NOESY using a mixing time of 80 and 100 ms. All spectra were processed using Topspin 4.0 and peaks picked using CARA 1.9.1 (Keller, 2004).

Biotin-LPS (B-LPS) Based ELISA

Black 96-well Greiner microplates were coated by incubating 100 μl 10 μg/mL SadA K9 in PBS-buffer (Cold spring harbor) overnight at 4° C. Wells were blocked the next day by incubating by 150 μL 2% bovine serum albumin (BSA) in PBS. 100 μL dilutions of Biotinylated-LPS ranging from 4 ng/mL to 0.06 ng/mL were added as a binding partner and incubated for 1 hour. Plates washed 3 times with 150 μL PBS+0.1% BSA. 100 μL 1:10 000 StrepTactin-conjugated horse radish peroxidase (IBA) for one hour, and developed with QuantaRed fluorescent substrate (Thermo) for 15 min and read fluorescence at Ex: 550 nm, Em: 610 nm.

Protocol:
1. Coat 96-well black Greiner/Nunc Maxisorp plate by adding 100 μL 10 μg/mL SadA fragment solution, leave overnight at 4° C.
2. Empty wells, block with 150 μL 5% BSA in PBS for 2 hours.
3. Wash 3 times with 150 μL 0.1% BSA in PBS
4. Empty wells, add 100 μL Biotinylated-LPS dilutions.
5. Wash 3 times with 150 μL 0.1% BSA in PBS
6. Add 100 μL Strep-Tactin conjugated HRP (IBA) (1:20,000 dilution in PBS+0.35 M NaCl, 50 mM MgSO4, 0.1% BSA) for 60 min
7. Wash 4 times with 150 μL PBS+0.35 M NaCl, 50 mM MgSo4, 0.1% BSA and then once with PBS+0.1% BSA.
8. Incubate with Quantared HRP-substrate, quench after 15 min.
9. Read fluorescence at Ex: 550 nm, Em: 610 nm.

All substrates were prepared following the instructions of the vendor. Where background was subtracted from signal, propagation of error was calculated by adding the individual standard deviations for the replicates to the baseline in quadrature (δQ=√(δa²+δb²+ . . . +δz²) where δQ is the uncertainty of a combination of sums Q). Error bars represent one standard deviation.

### Example 1—GCN4-PII Binds Lipid A

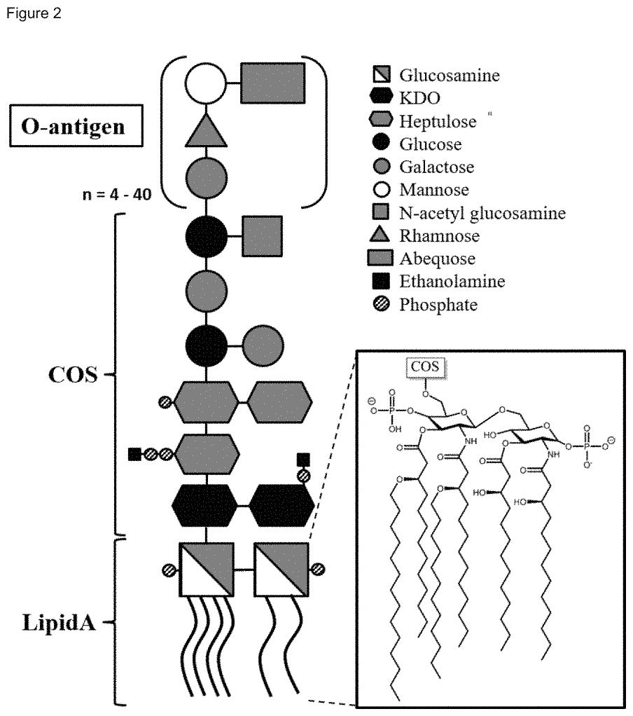

It was intended to investigate a putative interaction between LPS and two domains belonging to the trimeric autotransporter adhesin, SadA. Two earlier described SadA constructs (Alvarez et al., 2008; Hartmann et al., 2012), K9 and K14 were used, both stabilized by flanking GCN4-PII segments. K9 or K14 were covalently linked to a SPR-chip, and various LPS components injected. A schematic version of the structure of LPS is provided in FIG. 2 for reference.

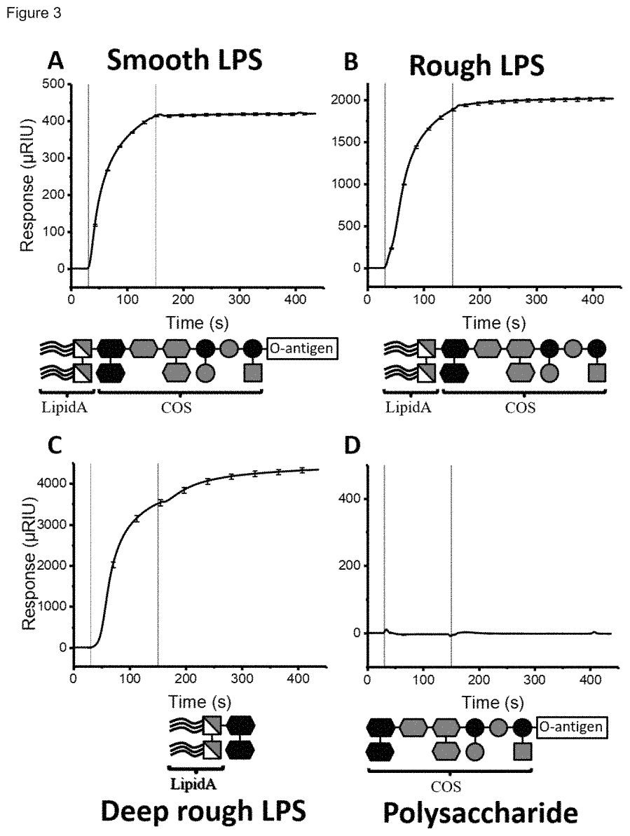

Injection of smooth LPS immediately gave a response, which approached a steady state towards the end of injection (FIG. 3a). During the following dissociation stage, the signal remained at the plateau, indicating that there was no off-rate. Injection of the rough and deep-rough LPS variants (FIGS. 3b and 3c), showed similar binding curves, except for a slight increase in signal during the dissociation phase, while the purified polysaccharide showed no binding characteristics (FIG. 3d).

The results showed that all variants containing the Lipid A moiety bound strongly to GCN4-PII, but the pure polysaccharide did not, thus localizing the interaction to the Lipid A moiety. However, the absent off-rate and the propensity of LPS to form aggregates in solution (Sasaki and White, 2008; Richter et al., 2011), meant complicated potential biophysical characterization of the interaction, and meant that the results could only be interpreted qualitatively. It was believed that the increase in signal following injection of the rough and deep-rough variants of LPS was inversely proportional to the number of sugar residues present in each variant. Particularly, deep rough LPS has a significantly higher hydrophobic to hydrophilic ratio, adopting a larger, less fluid morphology compared to LPS with longer sugar moieties (Richter et al., 2011). The signal increase following injection was thus interpreted as being due to a slower reorganization, and breakdown of the deep-rough aggregates compared to the smooth variant.

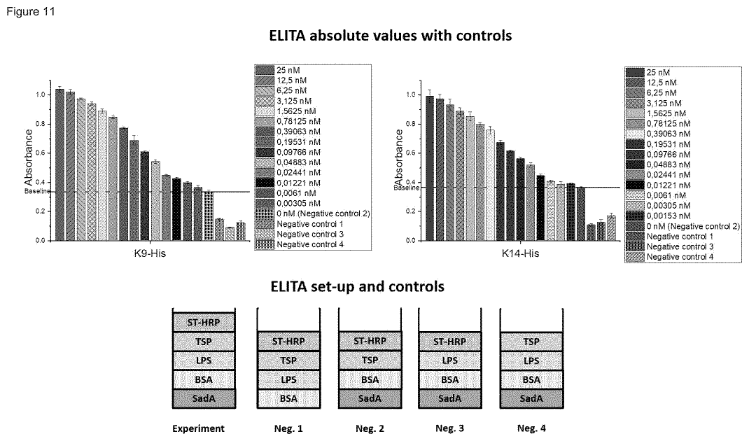

The constructs were purified using a 6×His-tag, which has been implicated to have an endotoxin depleting effect during purification due to unspecific binding (Mack et al., 2014). To evaluate the effect of the His-tag on binding, two GCN4-pII flanked SadA constructs which were identical except for the His-tag (K3, and K3-His) were compared. These yielded almost identical curves to each other and to the previous constructs, showing that the His-tag had no effect on binding (FIG. 10).

It was considered whether the nature of the interaction between GCN4-PII was hydrophobic, electrostatic, or a combination of both. The choice of regeneration solution helped to determine this. In the process of testing suitable regeneration buffers prior to the experiments, it was found that 1 M NaCl had no effect, whilst a mixture of non-denaturing detergents tallying to 0.3% regenerated the samples in less than 60 seconds. This indicated a strong hydrophobic factor involved in the interaction.

### Example 2—GNC4-PII Binds with High Affinity

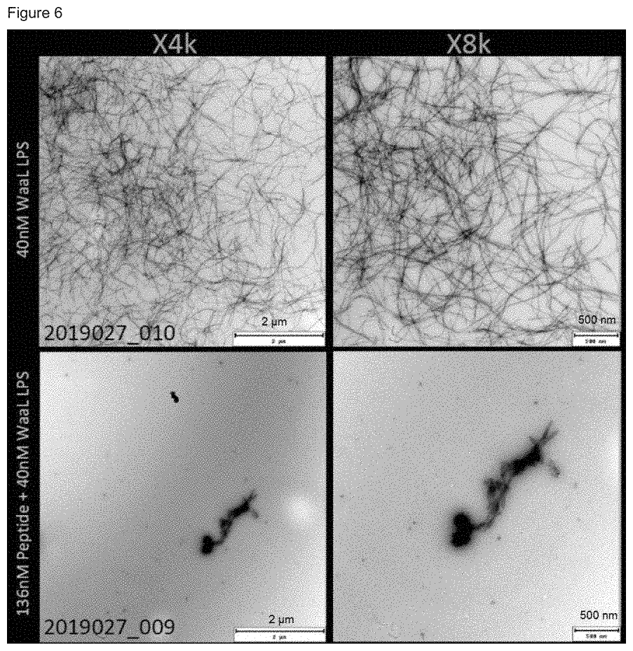

The SPR results were not suitable for determining the binding kinetics of the GCN4-pII/LPS interaction. In order to quantify the affinity, an ELISA-like tailspike adsorption (ELITA) assay described earlier (Schmidt et al., 2016) was modified by using purified proteins in lieu of whole bacteria. The assay was similar to a traditional ELISA, except that the antibody was replaced with a phage tailspike protein that recognized the O-antigen of LPS (FIG. 11). The results showed that both constructs exhibited an extremely high binding affinity in the lower pM range (FIG. 5), which is in concordance with the zero off-rates which were observed in the SPR experiments. This setup proved advantageous since it allowed for the use of LPS concentrations below the critical micelle concentration (CMC) of smooth LPS, which would otherwise have complicated the interpretation (Yu et al., 2006; Sasaki and White, 2008). However, due to the propensity of LPS to coat the microtiter wells prior to blocking, an indirect ligand-receptor interaction setup was not possible.

### Example 3—GCN4-pII Dissolves LPS Aggregates

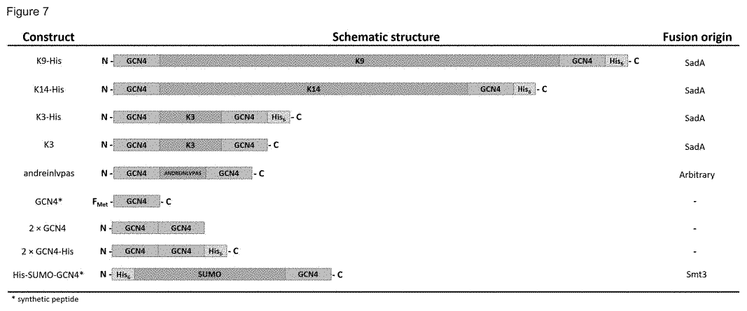

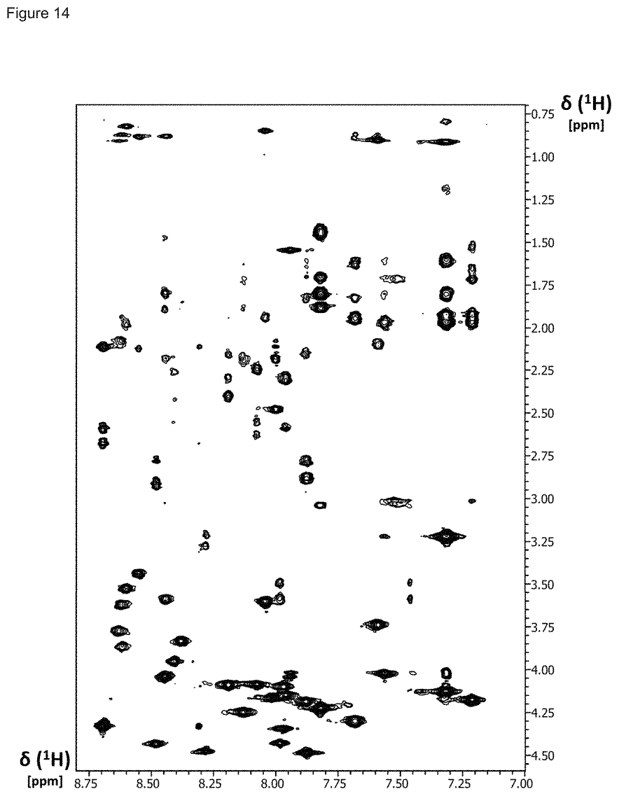

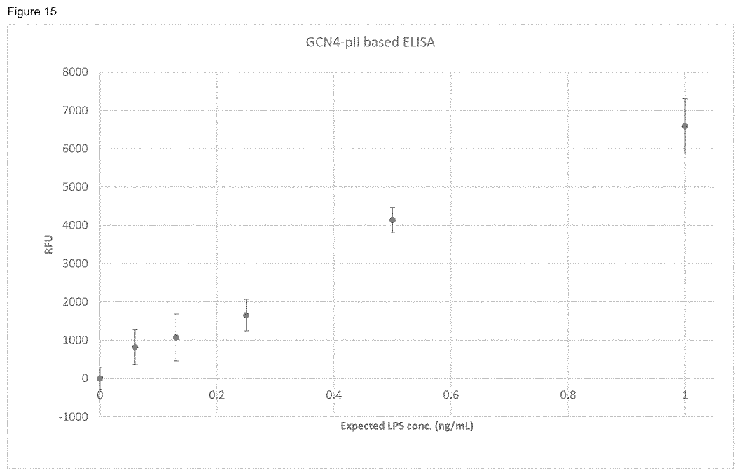

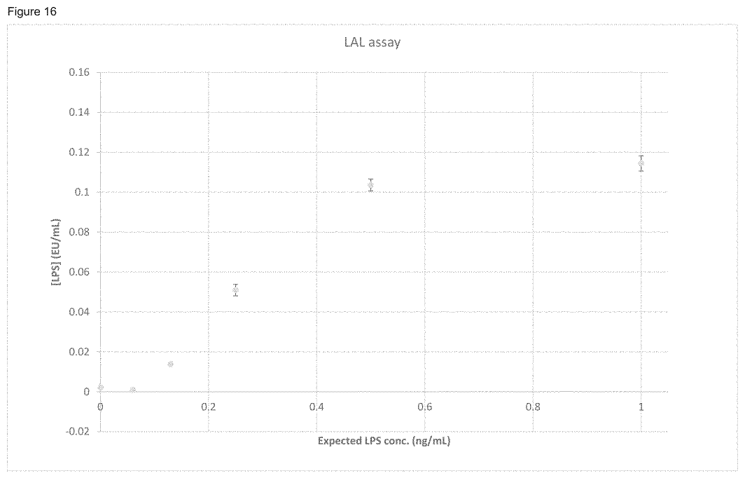

It was observed that adding GCN4-PII to LPS caused visible breakdown of the LPS aggregates. This was investigated by comparing the structures of rough LPS at different GCN4-pII ratios using transmission electron microscopy (FIG. 6). It was confirmed prior to the experiments that the synthetic GCN4-pII bound LPS and retained its α-helical structure upon binding using a LAL-masking assay (Schwarz et al., 2017), circular dichroism, and NMR. The NMR spectrum confirmed that the peptide existed in a homologous α-helical state (FIG. 14), which was retained upon LPS binding (FIG. 12), and showed at least 89% neutralizing effect (binding) on LPS at a GCN4-pII concentration of 1 μM (FIG. 13).

Rough LPS was observed with TEM to form tubular micelles with a radius of around 10 nm and lengths ranging up to hundreds of nm (FIG. 6, top), as reported earlier with cryo-EM (Richter et al., 2011; Broeker et al., 2018). Following incubation with equimolar GCN4-PII, the micellar structures completely disappeared, leaving occasional aggregates, probably caused by slight aggregation of the peptide-LPS complexes (FIG. 6, bottom).

Discussion of Results (Examples 1 to 3)

We originally set out to study a putative interaction between trimeric SadA domains and LPS. Our results however, show that the GCN4-pII adapters we used to stabilize our constructs displays an extremely high affinity for LPS. Interestingly, the affinity of GCN4-pII, with a K_{D} in the picomolar range, is 3-5 orders of magnitude higher than the human LPS immune receptors TLR4 (141 μM), CD14 (74 nM), MD-2 (2.33 μM), and LPS binding protein (3.5 nM). The dissociation constants we obtained with GCN4-pII are also 1-6 orders of magnitude higher than for polymxin B (48 μM), and even peptide avibodies specifically designed with the aim of highest achievable affinity. Furthermore, as opposed to several of the binding partners mentioned above, we have shown that GCN4-pII is specific to LipidA. We demonstrated that this interaction is reversible using detergents and that GCN4-pII readily dissolves LPS aggregates in solution, indicating that the interaction is largely hydrophobic. As far as we are aware, this is the first report of a trimeric coiled-coil motif binding LPS. GCN4-pII containing crystal structures earlier reported (Hartmann et al., 2012) show that the γ₂ and δ-carbons belonging to the core isoleucines protrude from the core, forming hydrophobic surfaces along the coiled-coil grooves. It is conceivable that one or more of the LipidA acyl chains can align along these grooves to form the extremely strong interaction, a model that also explains how GCN4-pII can break down LPS aggregates. However, GCN4-pII also has a C-terminal patch of cationic residues, and these may also contribute to the interaction.

### Example 4—Sensitivity of GCN4-pII Based ELISA

The aim of this experiment was to show that in principle the binding of the oligomeric protein to LPS, as shown here with GCN4-PII, could detect LPS quantities with equal or similar sensitivity to the LAL assay. As in previous examples, SadA-based constructs were used, in particular the K9 construct described above.

To ensure full reproducibility of the assay, the sensitivity experiment was conducted in 4 replicates with the final optimized conditions. In order to counteract the edge effect, only internal, randomized wells were used. The only exception was A3:A10, which was reserved for the highest concentration sample. The same samples were subsequently measured with the LAL assay for comparison. Only one replicate has been included in the results.

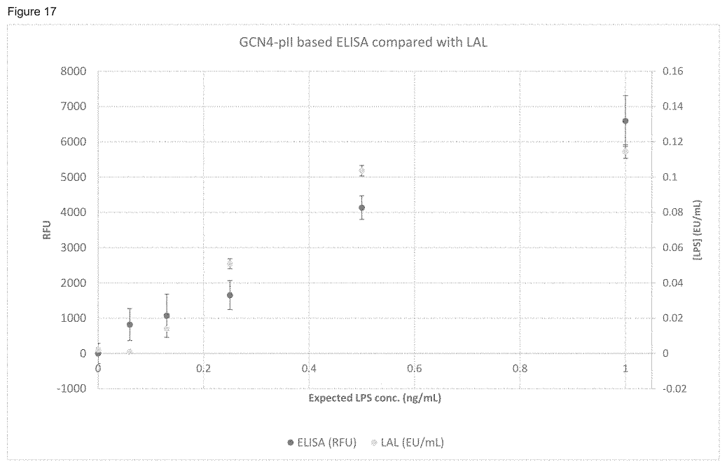

In the GCN4-pII based ELISA using biotinylated LPS (B-LPS) for detection (FIG. 15), a linear signal response was observed within the 0.06-1 ng/mL LPS concentration range, meaning that the assay could consistently detect B-LPS down to the lowest dilution (0.06 ng/mL).

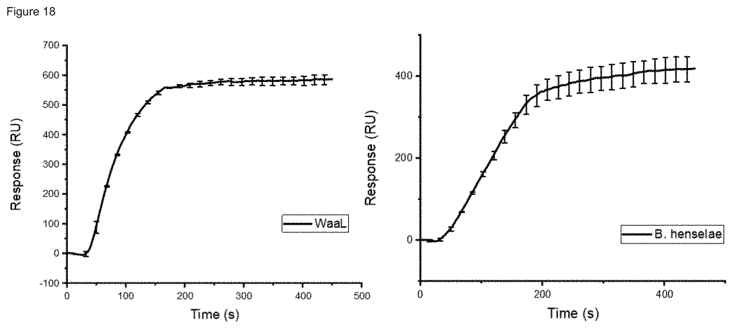

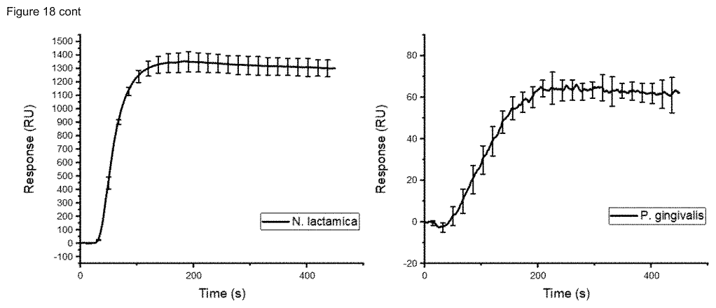

An LAL assay was also conducted for comparison. The concentration range of LPS used in the LAL assay was 0.01-0.1 EU/mL. The lowest dilution that gave a clear signal was 0.13 ng/mL (FIG. 16), meaning that the GNC4-pII based assay has comparable sensitivity to the LAL assay. A comparison of the results of the two assays is shown in FIG. 17.

### Example 5—Robustness of Binding Between GCN4-pII and LPS

To investigate the robustness of binding between GCN4-pII and different LPS types, SPR was used to check a broad selection of LPS-variants collected from various pathogens and proteobacteria (Table 4). In short, K9 was immobilized to a carboxyl matrix on the SPR-chip using EDC-NHS based amine coupling. Different LPS types were injected at 0.5 mg/mL in triplicates, to observe the signal.

TABLE 4

| Type | Candidate | Type | Note |
|---|---|---|---|
| α- | B. henselae | Rough | B. henselae LPS is one of few |
| proteobacteria | | | known non-pyrogenic LPS |
| | | | types (Chenoweth et al., |
| | | | 2004). |
| β- | Neisseria | Rough | Neisseria spp. Carry LPS-like |
| proteobacteria | lactamica | | molecules referred to as |
| | | | lipooligosaccharides (LOS). |
| | | | LOS are structurally very |
| | | | similar to classical LPS, but |
| | | | seldomly comes with O- |
| | | | antigen (Moran et al., 1996). |
| γ- | E. coli BL21, | — | Binding shown for several |
| proteobacteria | S. enterica, | | species earlier. |
| | S. anatum | | |
| Bacteroidetes | Porphyromonas | Smooth | Exact structure unknown, |
| | gingivalis | | probably smooth variant. |
| Unusual LPS | V. cholerae | Rough | V. cholerae LPS have unusual |
| | | | acylation-patterns of LipidA, |
| | | | and absence of phosphate |
| | | | group on the Kdo2-sugar |
| | | | (Reidl and Klose, 2002). |

In earlier work, we compared the binding of LPS sourced from γ-proteobacteria, namely S. enterica, S. anatum, and E. coli BL21. Injection of LPS immediately gave a response that approached a steady state towards the end of injection. During the following dissociation stage, the signal remained at the plateau, indicating that there was no measurable off-rate. Although the different curves had very similar shape, the final response (pRIU) varied between them with an inverse correlation to the amount of sugar moieties per LPS molecule. This means that rough LPS types (lacking the O-antigen) typically gave a significantly stronger signal compared to their smooth counterparts (with 0-antigen repeats). Since all LPS types were injected at the same gravimetric concentration (mg/mL), the difference in response probably reflected the lower molarity of the high molecular weight variants.

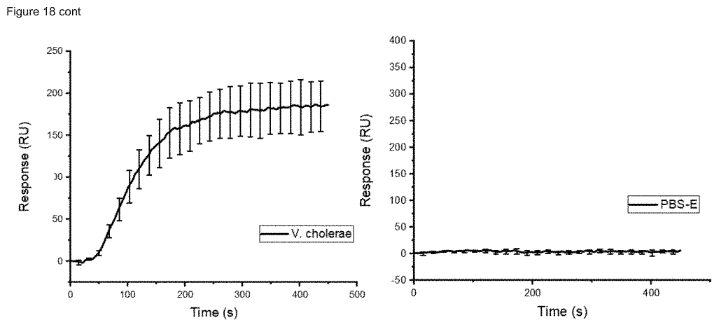

The binding curves of the LPS types we checked in our current work (FIG. 18) all share similar binding curves with no off-rate, indicating strong binding. S. typhimurium WaaL LPS is a rough type, expected to give a moderately high response upon injection. N. lactamica and B. henselae are both large rough variants, expected to give a response in the same range as WaaL, however, N. lactamica gave a response which was almost twice as large, which could be explained by a large amount of sialic acid modifications to the core-sugars. V. cholerae has a mixture of rough and low molecular weight smooth LPS. A response around ⅓ of the WaaL signal was therefore not unexpected. The P. gingivalis LPS had a low response, suggesting a medium-to-large smooth LPS type. The documentation coming with the commercially obtained P. gingivalis LPS did not name the strain or variant, so the weight will have to be confirmed by SDS-PAGE.

In conclusion, GCN4-pII bound to all LPS types that were tested.

Discussion of Results (Examples 4 and 5)

The LAL assay uses an enzymatic cascade in the blood of the horseshoe crab (Lee, 2007) that is highly sensitive to low amounts of LPS. Using the LAL assay in direct comparison, we were able to show that the GCN4-pII peptide can bind biotinylated LPS in concentrations that are barely detectable with the LAL assay, and that this binding still results in a visible signal when using routine detection methods for biotin coupled to a fluorescent enzyme substrate. Importantly, this detection method worked in different buffer backgrounds and also in an injectable drug background. We have achieved a sensitivity of 0.01 EU/mL LPS, which is comparable to the LAL assay, and our data suggests that even higher sensitivities can be achieved with our ELISA-like assay, e.g. by fine-tuning wash buffer conditions.

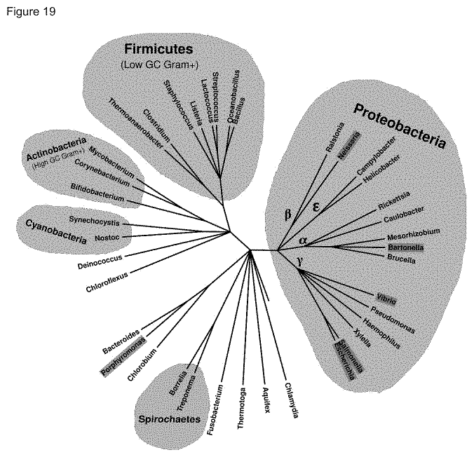

We used LPS variants from different clades of the proteobacteria, ranging from alpha- to gamma-proteobacteria, and from the Bacteroidetes (FIG. 19). Our selection covers Enteropathogens (Vibrio cholerae, Salmonella spp., Escherichia coli), intracellular pathogens (Bartonella henselae), and oral pathogens (Porphyromonas gingivalis), as well as commensal bacteria (Neisseria lactamica). One of the species used is known to have an LPS variant that do not elicit a strong immune response (Bartonella henselae) (Zahringer et al., 2004).

Salmonella spp. are located with Escherichia, and not displayed separately in the tree shown in FIG. 19. It is also noted that Bartonella spp. are closest to the displayed Brucella, and that Porphyromonas gingivalis is part of the Bacteroides for the purpose of this figure. Furthermore, the large groups of the Firmicutes, Actinobacteria, and Spirochaetes do not have LPS as part of their membrane components.

Overall, using SPR, we were able to show that all LPS variants used bound strongly to the GCN4-pII peptide. While there were visible differences in the “on rate” of binding, there was no detectable “off rate” for any of the LPS variants, suggesting that the peptide can detect all of these variants in a similar fashion (albeit with slight differences in binding kinetics).

### REFERENCES

Alvarez, B. H. et al. (2008) ‘A new expression system for protein crystallization using trimeric coiled-coil adaptors’, Protein Engineering Design & Selection, 21(1), pp. 11-18. doi: DOI 10.1093/protein/gzm071.
Andersson, K., Areskoug, D. and Hardenborg, E. (1999) ‘Exploring buffer space for molecular interactions’, Journal of Molecular Recognition, 12(5), pp. 310-315. doi: 10.1002/(SICI)1099-1352(199909/10)12:5<310::AID-JMR470>3.0.CO;2-5.
Bern, M., Goldberg, D., 2005. Automatic selection of representative proteins for bacterial phylogeny. BMC Evol. Biol.
Bertani, B. and Ruiz, N. (2018) ‘Function and Biogenesis of Lipopolysaccharides’, EcoSal Plus, 8(1). doi: 10.1128/ecosalplus.esp-0001-2018.
Broeker, N. K. et al. (2018) ‘In vitro studies of lipopolysaccharide-mediated DNA release of podovirus HK620’, Viruses. MDPI AG, 10(6), p. 289. doi: 10.3390/v10060289.
Chenoweth, M. R., Greene, C. E., Krause, D. C., Gherardini, F. C., 2004. Predominant outer membrane antigens of Balgonella henselae. Infect. Immun. 72, 3097-3105.
Darveau, R. P. and Hancock, R. E. W. (1983) ‘Procedure for isolation of bacterial lipopolysaccharides from both smooth and rough Pseudomonas aeruginosa and Salmonella typhimurium strains’, Journal of Bacteriology, 155(2), pp. 831-838.
Deiss, S. et al. (2014) ‘Your personalized protein structure: Andrei N. Lupas fused to GCN4 adaptors’, Journal of Structural Biology. Elsevier Inc., 186(3), pp. 380-385. doi: 10.1016/j.jsb.2014.01.013.
Delano, W. L. and Brunger, A. T. (1994) ‘Helix packing in proteins: Prediction and energetic analysis of dimeric, trimeric, and tetrameric GCN4 coiled coil structures’, Proteins: Structure, Function, and Bioinformatics. doi: 10.1002/prot. 340200202.
Fischer, M. J. E. (2010) ‘Amine Coupling Through EDC/NHS: A Practical Approach’, in. Humana Press, pp. 55-73. doi: 10.1007/978-1-60761-670-2_3.
Franke, D. et al. (2017) ‘ATSAS 2.8: A comprehensive data analysis suite for small-angle scattering from macromolecular solutions’, Journal of Applied Crystallography. doi: 10.1107/S1600576717007786.
Galanos, C., Lüderitz, O. and Westphal, 0. (1969) ‘A New Method for the Extraction of R Lipopolysaccharides’, European Journal of Biochemistry, 9(2), pp. 245-249. doi: 10.1111/j.1432-1033.1969.tb00601.x.
Harbury, P. B. et al. (1993) ‘A switch between two-, three-, and four-stranded coiled coils in GCN4 leucine zipper mutants’, Science, 262(5138), pp. 1401-1407. doi: 10.1126/science.8248779.
Hartmann, M. D. et al. (2012) ‘Complete fiber structures of complex trimeric autotransporter adhesins conserved in enterobacteria’, Proc Natl Acad Sci USA, 109(51), pp. 20907-20912. doi: 10.1073/pnas.1211872110.
Hitchcock, P. J. et al. (1986) ‘Lipopolysaccharide nomenclature—past, present, and future’, Journal of Bacteriology. doi: 10.1128/jb.166.3.699-705.1986.
Keller, R. (2004) The computer aided resonance assignment tutorial, Goldau, Switzerland: Cantina Verlag.
Lee, P. S., 2007. Endotoxins: pyrogens, LAL testing and depyrogenation, in: Williams, K. L. (Ed.)., Informa Healthcare, p. 419.
Lupas, A. N. and Gruber, M. (2005) ‘The structure of α-helical coiled coils’, Advances in Protein Chemistry. Academic Press Inc., 70, pp. 37-38. doi: 10.1016/50065-3233(05)70003-6.
Mack, L. et al. (2014) ‘Endotoxin depletion of recombinant protein preparations through their preferential binding to histidine tags’, Analytical Biochemistry. Academic Press, 466, pp. 83-88. doi: 10.1016/J.AB.2014.08.020.
Marolda, C. L. et al. (2006) ‘Micromethods for the Characterization of Lipid A-Core and O-Antigen Lipopolysaccharide’, in Glycobiology Protocols. New Jersey: Humana Press, pp. 237-252. doi: 10.1385/1-59745-167-3:237.
Moran, A. P., Prendergast, M. M., Appelmelk, B. J., 1996. Molecular mimicry of host structures by bacterial lipopolysaccharides and its contribution to disease. FEMS Immunol. Med. Microbiol. 16, 105-115.
Péterfi, Z. et al. (2000) ‘Comparison of Blocking Agents for an Elisa for Lps’, Journal of Immunoassay, 21(4). doi: 10.1080/01971520009349541.
Peterson, A. A. and McGroarty, E. J. (1985) ‘High-molecular-weight components in lipopolysaccharides of Salmonella typhimurium, Salmonella minnesota, and Escherichia coli’, Journal of Bacteriology, 162(2), pp. 738-745.
Raetz, C. R. H. and Whitfield, C. (2002a) ‘Lipopolysaccharide Endotoxins’, Annual Review of Biochemistry, 71(1), pp. 635-700. doi: 10.1146/annurev.biochem.71.110601.135414.
Raetz, C. R. H. and Whitfield, C. (2002b) ‘Lipopolysaccharide Endotoxins’, Annual Review of Biochemistry. Department of Biochemistry, Duke University Medical Center, Durham, N.C. 27710, USA. raetz@biochem.duke.edu, 71, pp. 635-700. doi: 10.1146/annurev.biochem.71.110601.135414.
Reidl, J., Klose, K. E., 2002. Vibrio cholerae and cholera: out of the water and into the host. FEMS Microbiol. Rev.
Richter, W. et al. (2011) ‘Morphology, size distribution, and aggregate structure of lipopolysaccharide and lipid A dispersions from enterobacterial origin.’, Innate immunity, 17(5), pp. 427-38. doi: 10.1177/1753425910372434.
Sasaki, H. and White, S. H. (2008) ‘Aggregation behavior of an ultra-pure lipopolysaccharide that stimulates TLR-4 receptors’, Biophysical Journal. Biophysical Society, 95(2), pp. 986-993. doi: 10.1529/biophysj.108.129197.
Schmidt, A. et al. (2016) ‘Bacteriophage tailspike protein based assay to monitor phase variable glucosylations in Salmonella O-antigens’. doi: 10.1186/s12866-016-0826-0.
Schwarz, H. et al. (2017) ‘Biological activity of masked endotoxin’, Scientific Reports. Nature Publishing Group, 7(1), pp. 1-11. doi: 10.1038/srep44750.
Studier, F. W. (2005) ‘Protein production by auto-induction in high density shaking cultures’, Protein Expr Purif. 2005/05/26. Biology Department, Brookhaven National Laboratory, Upton, N.Y. 11973, USA. studier@bnl.gov, 41(1), pp. 207-234.
Wishart, D. S. et al. (1995) ‘1H, 13C and 15N chemical shift referencing in biomolecular NMR’, Journal of Biomolecular NMR. doi: 10.1007/BF00211777.
Yu, L. et al. (2006) ‘Determination of critical micelle concentrations and aggregation numbers by fluorescence correlation spectroscopy: Aggregation of a lipopolysaccharide’, Analytica Chimica Acta, 556(1), pp. 216-225. doi: 10.1016/j.aca.2005.09.008.
Zähringer, U., Lindner, B., Knirel, Y. A., Van Den Akker, W. M. R., Hiestand, R., Heine, H., Dehio, C., 2004. Structure and biological activity of the short-chain lipopolysaccharide from Bartonella henselae ATCC 49882T. J. Biol. Chem.

1. Use of an oligomeric protein as a binding agent for binding to lipopolysaccharide (LPS), the oligomeric protein having a coiled coil structure comprising at least two monomer peptides, wherein each monomer peptide, which may be the same or different, is capable of forming an α-helix and comprises at least one core sequence having at least 60% sequence identity to the heptad repeat sequence of SEQ ID NO. 1.

2. The use of claim 1, wherein the core sequence comprises at least 3 heptad motifs a-b-c-d-e-f-g, or variants thereof, each variant comprising no more than 1 insertion or deletion to the heptad motif.

3. The use of claim 1 or claim 2, wherein at least 50% of the amino acid residues corresponding to positions a and d of the heptad motifs or variants thereof are hydrophobic residues.

4. The use of any one of claims 1 to 3, wherein the core sequence is flanked on one or both sides by a flanking amino acid sequence.

5. The use of claim 4, wherein the flanking sequence comprises one or more heptad motifs, and/or one or more parts thereof, preferably wherein the heptad motif in the flanking sequence corresponds to a heptad motif as found in SEQ ID NO. 1, or in a sequence having at least 80% sequence identity thereto, with the proviso that at least one of the amino acid residues a and d in the heptad motif is a hydrophobic residue.

6. The use of claim 4 or 5, wherein the flanking sequence comprises SEQ ID NO. 1, or a part thereof, or a sequence having at least 50% sequence identity thereto, wherein at least 50% of the amino acid residues corresponding to positions a and d of the heptad motifs of SEQ ID NO. 1, or variants thereof, are hydrophobic residues.

7. The use of any one of claims 3 to 6, wherein the flanking sequence comprises one or more linker sequences.

8. The use of any one of claims 1 to 7, wherein the monomer peptides each comprise two or more core sequences, wherein said core sequences may be the same or different.

9. The use of any one of claims 1 to 8, wherein the oligomeric protein is a dimer, trimer, or tetramer.

10. The use of any one of claims 1 to 9, wherein the oligomeric protein is a trimer.

11. The use of any one of claims 1 to 10, wherein the monomer peptides are provided as separate chains.

12. The use of any one of claims 1 to 10, wherein the monomer peptides are linked together.

13. The use of claim 12, wherein the monomer peptides are linked into a single chain or wherein the monomer peptides are linked by one or more chemical cross-links.

14. The use of any one of claims 1 to 13, wherein each hydrophobic residue in the heptad motifs or variants thereof is independently selected from the group consisting of leucine, isoleucine, valine, alanine, methionine, and chemical derivatives thereof.

15. The use of claim 14, wherein each hydrophobic residue is independently selected from leucine and isoleucine, or chemical derivatives thereof.

16. The use of claim 15, wherein the chemical derivatives are fluoroleucine or fluoroisoleucine.

17. The use of any one of claims 1 to 16, wherein at least 50% of the hydrophobic residues are isoleucine or fluoroisoleucine.

18. The use of any one of claims 1 to 17, wherein:
(i) at least 50% of the amino acid residues corresponding to positions b, c, e, f and g in the heptad repeats or variants thereof are polar residues; and/or
(ii) at least 5% of the amino acid residues corresponding to positions b, c, e, f and g in the heptad repeats or variants thereof are aliphatic residues.

19. The use of any one of claims 1 to 18, wherein each monomer peptide comprises 18 to 40 amino acids.

20. The use of any one of claims 1 to 19, wherein each monomer peptide comprises at least 4 cationic amino acids within the core sequence.

21. The use of any one of claims 1 to 20, wherein the oligomeric protein binds to LPS with a K_{D} in the nanomolar or lower size range.

22. The use of any one of claims 1 to 21, wherein the oligomeric protein is:
(i) in the form of a conjugate or fusion with one or more additional components;
(ii) immobilised on a solid substrate; or
(iii) conjugated to a directly detectable detection moiety.

23. The use of claim 22, wherein:
(i) the protein is conjugated with a detection moiety, an oligomerisation moiety or an immobilising moiety, or is in the form of a fusion protein with a fusion partner;
(ii) the protein is immobilised on a bead or resin, or in or on a well or vessel, or a column or filter material, or on a surface of a detection device; or
(iii) the detection moiety is a spectrophotometrically or spectroscopically detectable label.

24. The use of any one of claim 23, wherein the use of the oligomeric protein comprises detection and/or removal of LPS in or from a sample.

25. A method of binding LPS, the method comprising contacting the LPS, or a sample containing LPS, with an oligomeric protein as defined in any one of claims 1 to 23, to allow the protein to bind to the LPS to form a protein-lipopolysaccharide complex.

26. The method of claim 25, wherein the method further comprises detecting the presence of LPS in a sample, said method comprising:
(a) contacting the sample with an oligomeric protein as defined in any one of claims 1 to 23, to allow the protein to bind to the LPS to form a protein-lipopolysaccharide complex;
(b) detecting the presence of a protein-lipopolysaccharide complex.

27. The method of claim 25, wherein the method further comprises removing LPS from a sample, said method comprising:
(a) contacting the sample with an oligomeric protein as defined in any one of claims 1 to 23, to allow the protein to bind to the LPS to form a protein-lipopolysaccharide complex;
(b) separating the peptide-lipopolysaccharide complex from the sample.

28. The method of any one of claims 25 to 27, wherein the oligomeric protein is in the form of a conjugate comprising a detectable label and/or wherein the oligomeric protein is immobilised on a solid substrate.

29. The method of any one of claims 25 to 28, wherein the sample is a clinical sample derived from a patient or a sample of a product for testing for endotoxin contamination.

30. The method of claim 29, wherein the sample is a blood sample, or a sample derived from a blood sample.

31. A kit comprising;
(i) an oligomeric protein as defined in any one of claims 1 to 17; and
(ii) at least one non-denaturing detergent.

32. The kit of claim 31, wherein the kit is for use according to any one of claims 1 to 24, or in the method of any one of claims 25 to 30.

33. A product comprising an oligomeric protein immobilised on a solid substrate, wherein the oligomeric protein is as defined in any one of claims 1 to 21.
